(19) United States
(12) Patent Application Publication
(10) Pub. No.: US 2014/0275194 A1
(43) Pub. Date: Sep. 18, 2014

(54) FILMS AND DRUG DELIVERY SYSTEMS FOR RIZATRIPTAN

(71) Applicant: MonoSol Rx, LLC, Warren, NJ (US)

(72) Inventors: Eric Dadey, Furlong, PA (US); Alexander Mark Schobel, Whitehouse Station, NJ (US); Daniel R. Barber, Summit, NJ (US); Garry L. Myers, Kingsport, TN (US)

(21) Appl. No.: 13/844,689

(22) Filed: Mar. 15, 2013

(51) Int. Cl. A61K 31/4196

(52) U.S. Cl. CPC A61K 31/4196
USPC 514/383

(57) ABSTRACT

The invention relates to film products including a polymer component and a therapeutically effective amount of rizatriptan or a pharmaceutically acceptable salt thereof. The invention further relates to film products including a polymer component and a therapeutically effective amount of rizatriptan benzoate.

### FIELD OF THE INVENTION

The invention relates to rapidly dissolving films and methods of their preparation. The films contain a polymer component. The films may also contain an active ingredient that is evenly distributed throughout the film. The even or uniform distribution is achieved by controlling one or more parameters, and particularly the elimination of air pockets prior to and during film formation and the use of a drying process that reduces aggregation or conglomeration of the active or the components in the film as it forms into a solid structure.

### BACKGROUND OF THE RELATED TECHNOLOGY

Active ingredients, such as drugs or pharmaceuticals, may be prepared in a tablet form to allow for accurate and consistent dosing. However, this form of preparing and dispensing medications has many disadvantages including that a large proportion of adjuvants that must be added to obtain a size able to be handled, that a larger medication form requires additional storage space, and that dispensing includes counting the tablets which has a tendency for inaccuracy. In addition, many persons, estimated to be as much as 28% of the population, have difficulty swallowing tablets. While tablets may be broken into smaller pieces or even crushed as a means of overcoming swallowing difficulties, this is not a suitable solution for many tablet or pill forms. For example, crushing or destroying the tablet or pill form to facilitate ingestion, alone or in admixture with food, may also destroy the controlled release properties.

As an alternative to tablets and pills, films may be used to carry active ingredients such as drugs, pharmaceuticals, and the like. However, historically films and the process of making drug delivery systems therefrom have suffered from a number of unfavorable characteristics that have not allowed them to be used in practice.

Films that incorporate a pharmaceutically active ingredient are disclosed in expired U.S. Pat. No. 4,136,145 to Fuchs, et al. (“Fuchs”). These films may be formed into a sheet, dried and then cut into individual doses. The Fuchs disclosure alleges the fabrication of a uniform film, which includes the combination of water-soluble polymers, surfactants, flavors, sweeteners, plasticizers and drugs. These allegedly flexible films are disclosed as being useful for oral, topical or enteral use. Examples of specific uses disclosed by Fuchs include application of the films to mucosal membrane areas of the body, including the mouth, rectal, vaginal, nasal and ear areas.

Examination of films made in accordance with the process disclosed in Fuchs, however, reveals that such films suffer from the aggregation or conglomeration of particles, i.e., self-aggregation, making them inherently non-uniform. This result can be attributed to Fuchs' process parameters, which although not disclosed likely include the use of relatively long drying times, thereby facilitating intermolecular attractive forces, convection forces, air flow and the like to form such agglomeration.

The formation of agglomerates randomly distributes the film components and any active present as well. When large dosages are involved, a small change in the dimensions of the film would lead to a large difference in the amount of active per film. If such films were to include low dosages of active, it is possible that portions of the film may be substantially devoid of any active. Since sheets of film are usually cut into unit doses, certain doses may therefore be devoid of or contain an insufficient amount of active for the recommended treatment. Failure to achieve a high degree of accuracy with respect to the amount of active ingredient in the cut film can be harmful to the patient. For this reason, dosage forms formed by processes such as Fuchs, would not likely meet the stringent standards of governmental or regulatory agencies, such as the U.S. Food and Drug Administration (“FDA”), relating to the variation of active in dosage forms. Currently, as required by various world regulatory authorities, dosage forms may not vary more than 10% from a desired or label amount in the amount of active present. When applied to dosage units based on films, this virtually mandates that uniformity in the film be present.

The problems of self-aggregation leading to non-uniformity of a film were addressed in U.S. Pat. No. 4,849,246 to Schmidt (“Schmidt”). Schmidt specifically pointed out that the methods disclosed by Fuchs did not provide a uniform film and recognized that that the creation of a non-uniform film necessarily prevents accurate dosing, which as discussed above is especially important in the pharmaceutical area. Schmidt abandoned the idea that a mono-layer film, such as described by Fuchs, may provide an accurate dosage form and instead attempted to solve this problem by forming a multi-layered film. Moreover, his process is a multi-step process that adds expense and complexity and is not practical for commercial use.

Other U.S. patents directly addressed the problems of particle self-aggregation and non-uniformity inherent in conventional film forming techniques. In one attempt to overcome non-uniformity, U.S. Pat. No. 5,629,003 to Horstmann et al. and U.S. Pat. No. 5,948,430 to Zerbe et al. incorporated additional ingredients, i.e. gel formers and polyhydric alcohols respectively, to increase the viscosity of the film prior to drying in an effort to reduce aggregation of the components in the film. These methods have the disadvantage of requiring additional components, which translates to additional cost and manufacturing steps. Furthermore, both methods employ the use the conventional time-consuming drying methods such as a high-temperature air-bath using a drying oven, drying tunnel, vacuum drier, or other such drying equipment. The long length of drying time aids in promoting the aggregation of the active and other adjuvant, notwithstanding the use of viscosity modifiers. Such processes also run the risk of exposing the active, i.e., a drug, or vitamin C, or other components to prolonged exposure to moisture and elevated temperatures, which may render it ineffective or even harmful.

In addition to the concerns associated with degradation of an active during extended exposure to moisture, the conventional drying methods themselves are unable to provide uniform films. The length of heat exposure during conventional processing, often referred to as the “heat history”, and the manner in which such heat is applied, have a direct effect on the formation and morphology of the resultant film product. Uniformity is particularly difficult to achieve via conventional drying methods where a relatively thicker film, which is well-suited for the incorporation of a drug active, is desired. Thicker uniform films are more difficult to achieve because the surfaces of the film and the inner portions of the film do not experience the same external conditions simultaneously during drying. Thus, observation of relatively thick films made from such conventional processing shows a non-uniform structure caused by convection and intermolecular forces and requires greater than 10% moisture to remain flexible. The amount of free moisture can often interfere over time with the drug leading to potency issues and therefore inconsistency in the final product.

Conventional drying methods generally include the use of forced hot air using a drying oven, drying tunnel, and the like. The difficulty in achieving a uniform film is directly related to the rheological properties and the process of water evaporation in the film-forming composition. When the surface of an aqueous polymer solution is contacted with a high temperature air current, such as a film-forming composition passing through a hot air oven, the surface water is immediately evaporated forming a polymer film or skin on the surface. This seals the remainder of the aqueous film-forming composition beneath the surface, forming a barrier through which the remaining water must force itself as it is evaporated in order to achieve a dried film. As the temperature outside the film continues to increase, water vapor pressure builds up under the surface of the film, stretching the surface of the film, and ultimately ripping the film surface open allowing the water vapor to escape. As soon as the water vapor has escaped, the polymer film surface reforms, and this process is repeated, until the film is completely dried. The result of the repeated destruction and reformation of the film surface is observed as a “ripple effect” which produces an uneven, and therefore non-uniform film. Frequently, depending on the polymer, a surface will seal so tightly that the remaining water is difficult to remove, leading to very long drying times, higher temperatures, and higher energy costs.

Other factors, such as mixing techniques, also play a role in the manufacture of a pharmaceutical film suitable for commercialization and regulatory approval. Air can be trapped in the composition during the mixing process or later during the film making process, which can leave voids in the film product as the moisture evaporates during the drying stage. The film frequently collapse around the voids resulting in an uneven film surface and therefore, non-uniformity of the final film product. Uniformity is still affected even if the voids in the film caused by air bubbles do not collapse. This situation also provides a non-uniform film in that the spaces, which are not uniformly distributed, are occupying area that would otherwise be occupied by the film composition. None of the above-mentioned patents either addresses or proposes a solution to the problems caused by air that has been introduced to the film.

Therefore, there is a need for methods and compositions for film products, which use a minimal number of materials or components, and which provide a substantially non-self-aggregating uniform heterogeneity throughout the area of the films. Desirably, such films are produced through a selection of a polymer or combination of polymers that will provide a desired viscosity, a film-forming process such as reverse roll coating, and a controlled, and desirably rapid, drying process which serves to maintain the uniform distribution of non-self-aggregated components without the necessary addition of gel formers or polyhydric alcohols and the like which appear to be required in the products and for the processes of prior patents, such as the aforementioned Horstmann and Zerbe patents. Desirably, the films will also incorporate compositions and methods of manufacture that substantially reduce or eliminate air in the film, thereby promoting uniformity in the final film product.

### SUMMARY OF THE INVENTION

One embodiment of the present invention is a film product including a polymer component and a therapeutically effective amount of a triptan or a pharmaceutically acceptable salt thereof.

One embodiment of the present invention is a film product including a polymer component and a therapeutically effective amount of rizatriptan or a pharmaceutically acceptable salt thereof.

Another embodiment of the present invention is film product including a polymer component and a therapeutically effective amount of rizatriptan benzoate.

The present invention is also directed to processes for manufacturing pharmaceutical and bioactive active containing films, suitable for commercialization and U.S. Food and Drug Administration (“FDA”) approval.

The present invention is also directed to processes for making a film having a substantial uniform distribution of components such that the uniformity of content in the amount pharmaceutical and/or cosmetic active in an individual dosage unit as sampled from the film varies no more than about 10% or less percent difference; and desirably varies no more than about 9% or less percent difference, more desirably varies no more than about 8% or less percent difference, even more desirably varies no more than about 7% or less percent difference, even more desirably varies no more than about 6% or less percent difference, even more desirably varies no more than about 5% or less percent difference, even more desirably varies no more than about 4% or less percent difference, even more desirably varies no more than about 3% or less percent difference, even more desirably varies no more than about 2% or less percent difference, even more desirably varies no more than about 1% or less percent difference, even more desirably varies no more than about 0.5% or less percent difference.

The present invention is also directed to processes for making a film having a substantial uniform distribution of components such that content uniformity in amount of active in individual dosage units sampled from two or more lots of film such that the amount of active varies no more than about 10% or less from the desired or target or labeled amount; and desirably varies no more than about 9% or less, more desirably varies no more than about 8% or less, even more desirably varies no more than about 7% or less, even more desirably varies no more than about 6% or less, even more desirably varies no more than about 5% or less, even more desirably varies no more than about 4% or less, even more desirably varies no more than about 3% or less, even more desirably varies no more than about 2% or less, even more desirably varies no more than about 1% or less, even more desirably varies no more than about 0.5% or less, all of these cascading amounts again from a desired or target or labeled amount.

The present invention is also directed to processes where one manufactured lot of film can contain approximately 500,000-2,000,000 or more individual dosage units.

The present invention is also directed to products and processes wherein content uniformity in amount of active is indicated or determined through analytical chemical tests, such as High Performance Liquid Chromatography (HPLC).

### BRIEF DESCRIPTION OF THE DRAWINGS

FIG. 1 shows a side view of a package containing a unit dosage film of the present invention.

FIG. 2 shows a top view of two adjacently coupled packages containing individual unit dosage forms of the present invention, separated by a tearable perforation.

FIG. 3 shows a side view of the adjacently coupled packages of FIG. 2 arranged in a stacked configuration.

FIG. 4 shows a perspective view of a dispenser for dispensing the packaged unit dosage forms, dispenser containing the packaged unit dosage forms in a stacked configuration.

FIG. 5 is a schematic view of a roll of coupled unit dose packages of the present invention.

FIG. 6 is a schematic view of an apparatus suitable for preparation of a pre-mix, addition of an active, and subsequent formation of the film.

FIG. 7 is a schematic view of an apparatus suitable for drying the films of the present invention.

FIG. 8 is a sequential representation of the drying process of the present invention.

FIG. 9 is a photographic representation of a film dried by conventional drying processes.

FIG. 10 is a photographic representation of a film dried by conventional drying processes.

FIG. 11 is a photographic representation of a film dried by conventional drying processes.

FIG. 12 is a photographic representation of a film dried by conventional drying processes.

FIG. 13 is a photographic representation of a film dried by conventional drying processes.

FIG. 14 is a photographic representation of a film dried by conventional drying processes.

FIG. 15 is a photographic representation of a film dried by conventional drying processes.

FIG. 16 is a photographic representation of a film dried by conventional drying processes.

FIG. 17 is a photographic representation of a film dried by the inventive drying process.

FIG. 18 is a photomicrographic representation of a film containing fat coated particles dried by the inventive drying process.

FIG. 19 is a photomicrographic representation of a film containing fat coated particles dried by the inventive drying process.

FIG. 20 is a photomicrographic representation of a film containing fat coated particles dried by the inventive drying process.

FIG. 21 is a photomicrographic representation of a film containing fat coated particles dried by the inventive drying process.

FIG. 22 is a photomicrographic representation of a film containing fat coated particles dried by the inventive drying process.

FIG. 23 is a photomicrographic representation of a film containing fat coated particles dried by the inventive drying process.

FIG. 24 is a photomicrographic representation of a film containing fat coated particles dried by the inventive drying process.

FIG. 25 is a photomicrographic representation of a film containing fat coated particles dried by the inventive drying process.

FIG. 26 is a photomicrographic representation of fat coated particles not in film, heated for 9 minutes at 80° C.

FIG. 27 is a photomicrographic representation of fat coated particles not in film, heated for 9 minutes at 80° C.

FIG. 28 is a photomicrographic representation of fat coated particles at room temperature prior to processing.

FIG. 29 is a photomicrographic representation of fat coated particles at room temperature prior to processing.

FIG. 30 is a photomicrographic representation of fat coated particles at room temperature prior to processing.

FIG. 31 is a photomicrographic representation of fat coated particles at room temperature prior to processing.

FIG. 32 is a graphical representation of a microarray on the blood of a human after ingestion by the human of a film of the present invention containing a bovine derived protein.

FIG. 33 is a graphical representation of the temperature differential between the inside and outside of a film of the present invention during drying.

FIG. 34 is a graphical representation of the temperature differential between the inside and outside of a film of the present invention during drying.

FIG. 35 is a schematic representation of a continuously-linked zone drying apparatus in accordance with the present invention.

FIG. 36 is a schematic representation of a separate zone drying apparatus in accordance with the present invention.

FIG. 37 is a schematic representation of a extrusion device for use in producing films of the present invention.

FIG. 38 shows plasma rizatriptan levels following oral administration of 10 mg MAXALT® Tablet to Göttengin Minipigs.

FIG. 39 shows plasma rizatriptan levels following sublingual application of the a 10 mg rizatriptan film of the present invention to Göttengin Minipig.

FIG. 40 shows a comparison of the average plasma rizatripan levels between 10 mg MAXALT Oral Tablet and a 10 mg raizatriptan sublingual film of the present invention.

FIG. 41 shows the mean plasma rizatriptan concentration on a linear scale, from time 0 to 12 hours after dosing for MAXALT Oral Tablet and a 10 mg raizatriptan sublingual film of the present invention.

### DETAILED DESCRIPTION OF THE INVENTION

For the purposes of the present invention the term non-self-aggregating uniform heterogeneity refers to the ability of the films of the present invention, which are formed from one or more components in addition to a polar solvent, to provide a substantially reduced occurrence of, i.e. little or no, aggregation or conglomeration of components within the film as is normally experienced when films are formed by conventional drying methods such as a high-temperature air-bath using a drying oven, drying tunnel, vacuum drier, or other such drying equipment. The term heterogeneity, as used in the present invention, includes films that will incorporate a single component, such as a polymer, as well as combinations of components, such as a polymer and an active. Uniform heterogeneity includes the substantial absence of aggregates or conglomerates as is common in conventional mixing and heat drying methods used to form films.

Furthermore, the films of the present invention have a substantially uniform thickness, which is also not provided by the use of conventional drying methods used for drying water-based polymer systems. The absence of a uniform thickness detrimentally affects uniformity of component distribution throughout the area of a given film.

The film products of the present invention are produced by a combination of a properly selected polymer and a polar solvent, optionally including an active ingredient as well as other fillers known in the art. These films provide a non-self-aggregating uniform heterogeneity of the components within them by utilizing a selected casting or deposition method and a controlled drying process. Examples of controlled drying processes include, but are not limited to, the use of the apparatus disclosed in U.S. Pat. No. 4,631,837 to Magoon (“Magoon”), herein incorporated by reference, as well as hot air impingement across the bottom substrate and bottom heating plates. Another drying technique for obtaining the films of the present invention is controlled radiation drying, in the absence of uncontrolled air currents, such as infrared and radio frequency radiation (i.e. microwaves).

The objective of the drying process is to provide a method of drying the films that avoids complications, such as the noted “rippling” effect, that are associated with conventional drying methods and which initially dry the upper surface of the film, trapping moisture inside. In conventional oven drying methods, as the moisture trapped inside subsequently evaporates, the top surface is altered by being ripped open and then reformed. These complications are avoided by the present invention, and a uniform film is provided by drying the bottom surface of the film first or otherwise preventing the formation of polymer film formation (skin) on the top surface of the film prior to drying the depth of the film. This may be achieved by applying heat to the bottom surface of the film with substantially no top air flow, or alternatively by the introduction of controlled microwaves to evaporate the water or other polar solvent within the film, again with substantially no top air flow. Yet alternatively, drying may be achieved by using balanced fluid flow, such as balanced air flow, where the bottom and top air flows are controlled to provide a uniform film. In such a case, the air flow directed at the top of the film should not create a condition which would cause movement of particles present in the wet film, due to forces generated by the air currents. Additionally, air currents directed at the bottom of the film should desirably be controlled such that the film does not lift up due to forces from the air. Uncontrolled air currents, either above or below the film, can create non-uniformity in the final film products. The humidity level of the area surrounding the top surface may also be appropriately adjusted to prevent premature closure or skinning of the polymer surface.

This manner of drying the films provides several advantages. Among these are the faster drying times and a more uniform surface of the film, as well as uniform distribution of components for any given area in the film. In addition, the faster drying time allows viscosity to quickly build within the film, further encouraging a uniform distribution of components and decrease in aggregation of components in the final film product. Desirably, the drying of the film will occur within about ten minutes or fewer, or more desirably within about five minutes or fewer.

The present invention yields exceptionally uniform film products when attention is paid to reducing the aggregation of the compositional components. By avoiding the introduction of and eliminating excessive air in the mixing process, selecting polymers and solvents to provide a controllable viscosity and by drying the film in a rapid manner from the bottom up, such films result.

The products and processes of the present invention rely on the interaction among various steps of the production of the films in order to provide films that substantially reduce the self-aggregation of the components within the films. Specifically, these steps include the particular method used to form the film, making the composition mixture to prevent air bubble inclusions, controlling the viscosity of the film forming composition and the method of drying the film. More particularly, a greater viscosity of components in the mixture is particularly useful when the active is not soluble in the selected polar solvent in order to prevent the active from settling out. However, the viscosity must not be too great as to hinder or prevent the chosen method of casting, which desirably includes reverse roll coating due to its ability to provide a film of substantially consistent thickness.

In addition to the viscosity of the film or film-forming components or matrix, there are other considerations taken into account by the present invention for achieving desirable film uniformity. For example, stable suspensions are achieved which prevent solid (such as drug particles) sedimentation in non-colloidal applications. One approach provided by the present invention is to balance the density of the particulate (ρₚ) and the liquid phase (ρ₁) and increase the viscosity of the liquid phase (μ). For an isolated particle, Stokes law relates the terminal settling velocity (Vo) of a rigid spherical body of radius (r) in a viscous fluid, as follows:

Vₒ=(2gr^{r})(ρₚ−ρₗ)/9μ,

At high particle concentrations, however, the local particle concentration will affect the local viscosity and density. The viscosity of the suspension is a strong function of solids volume fraction, and particle-particle and particle-liquid interactions will further hinder settling velocity.

Stokian analyses has shown that the incorporation of a third phase, dispersed air or nitrogen, for example, promotes suspension stability. Further, increasing the number of particles leads to a hindered settling effect based on the solids volume fraction. In dilute particle suspensions, the rate of sedimentation, v, can be expressed as:

v/Vₒ=1/(1+κΦ)

where κ=a constant, and φ is the volume fraction of the dispersed phase. More particles suspended in the liquid phase results in decreased velocity. Particle geometry is also an important factor since the particle dimensions will affect particle-particle flow interactions.

Similarly, the viscosity of the suspension is dependent on the volume fraction of dispersed solids. For dilute suspensions of non-interaction spherical particles, an expression for the suspension viscosity can be expressed as:

μ/μₒ=1+2.50

where μₒ is the viscosity of the continuous phase and φ is the solids volume fraction. At higher volume fractions, the viscosity of the dispersion can be expressed as

μ/μₒ=1+2.5φ+C₁φ²+C₂φ³+ . . .

where C is a constant.

The viscosity of the liquid phase is critical and is desirably modified by customizing the liquid composition to a viscoelastic non-Newtonian fluid with low yield stress values. This is the equivalent of producing a high viscosity continuous phase at rest. Formation of a viscoelastic or a highly structured fluid phase provides additional resistive forces to particle sedimentation. Further, flocculation or aggregation can be controlled minimizing particle-particle interactions. The net effect would be the preservation of a homogeneous dispersed phase.

The addition of hydrocolloids to the aqueous phase of the suspension increases viscosity, may produce viscoelasticity and can impart stability depending on the type of hydrocolloid, its concentration and the particle composition, geometry, size, and volume fraction. The particle size distribution of the dispersed phase needs to be controlled by selecting the smallest realistic particle size in the high viscosity medium, i.e., <500 μm. The presence of a slight yield stress or elastic body at low shear rates may also induce permanent stability regardless of the apparent viscosity. The critical particle diameter can be calculated from the yield stress values. In the case of isolated spherical particles, the maximum shear stress developed in settling through a medium of given viscosity can be given as

τₘₐₓ=3Vμ/2r

For pseudoplastic fluids, the viscosity in this shear stress regime may well be the zero shear rate viscosity at the Newtonian plateau.

A stable suspension is an important characteristic for the manufacture of a pre-mix composition which is to be fed into the film casting machinery film, as well as the maintenance of this stability in the wet film stage until sufficient drying has occurred to lock-in the particles and matrix into a sufficiently solid form such that uniformity is maintained. For viscoelastic fluid systems, a rheology that yields stable suspensions for extended time period, such as 24 hours, must be balanced with the requirements of high-speed film casting operations. A desirable property for the films is shear thinning or pseudoplasticity, whereby the viscosity decreases with increasing shear rate. Time dependent shear effects such as thixotropy are also advantageous. Structural recovery and shear thinning behavior are important properties, as is the ability for the film to self-level as it is formed.

The rheology requirements for the inventive compositions and films are quite severe. This is due to the need to produce a stable suspension of particles, for example 30-60 wt %, in a viscoelastic fluid matrix with acceptable viscosity values throughout a broad shear rate range. During mixing, pumping, and film casting, shear rates in the range of 10-10⁵ sec.⁻¹ may be experienced and pseudoplasticity is the preferred embodiment.

In film casting or coating, rheology is also a defining factor with respect to the ability to form films with the desired uniformity. Shear viscosity, extensional viscosity, viscoelasticity, structural recovery will influence the quality of the film. As an illustrative example, the leveling of shear-thinning pseudoplastic fluids has been derived as

α^{(n-1/n)}=αₒ^{(n-1/n)}−((n−1)/(2n−1))(τ/K)^{1/n}(2π/λ)^{(3+n)/n}h^{(2n+1)/n}t

where α is the surface wave amplitude, αₒ is the initial amplitude, λ is the wavelength of the surface roughness, and both “n” and “K” are viscosity power law indices. In this example, leveling behavior is related to viscosity, increasing as n decreases, and decreasing with increasing K.

Desirably, the films or film-forming compositions of the present invention have a very rapid structural recovery, i.e. as the film is formed during processing, it doesn't fall apart or become discontinuous in its structure and compositional uniformity. Such very rapid structural recovery retards particle settling and sedimentation. Moreover, the films or film-forming compositions of the present invention are desirably shear-thinning pseudoplastic fluids. Such fluids with consideration of properties, such as viscosity and elasticity, promote thin film formation and uniformity.

Thus, uniformity in the mixture of components depends upon numerous variables. As described herein, viscosity of the components, the mixing techniques and the rheological properties of the resultant mixed composition and wet casted film are important aspects of the present invention. Additionally, control of particle size and particle shape are further considerations. Desirably, the size of the particulate a particle size of 150 microns or less, for example 100 microns or less. Moreover, such particles may be spherical, substantially spherical, or non-spherical, such as irregularly shaped particles or ellipsoidally shaped particles. Ellipsoidally shaped particles or ellipsoids are desirable because of their ability to maintain uniformity in the film forming matrix as they tend to settle to a lesser degree as compared to spherical particles.

A number of techniques may be employed in the mixing stage to prevent bubble inclusions in the final film. To provide a composition mixture with substantially no air bubble formation in the final product, anti-foaming or surface-tension reducing agents are employed. Additionally, the speed of the mixture is desirably controlled to prevent cavitation of the mixture in a manner which pulls air into the mix. Finally, air bubble reduction can further be achieved by allowing the mix to stand for a sufficient time for bubbles to escape prior to drying the film. Desirably, the inventive process first forms a masterbatch of film-forming components without active ingredients such as drug particles or volatile materials such as flavor oils. The actives are added to smaller mixes of the masterbatch just prior to casting. Thus, the masterbatch pre-mix can be allowed to stand for a longer time without concern for instability in drug or other ingredients.

When the matrix is formed including the film-forming polymer and polar solvent in addition to any additives and the active ingredient, this may be done in a number of steps. For example, the ingredients may all be added together or a pre-mix may be prepared. The advantage of a pre-mix is that all ingredients except for the active may be combined in advance, with the active added just prior to formation of the film. This is especially important for actives that may degrade with prolonged exposure to water, air or another polar solvent.

FIG. 6 shows an apparatus 20 suitable for the preparation of a pre-mix, addition of an active and subsequent formation of a film. The pre-mix or master batch 22, which includes the film-forming polymer, polar solvent, and any other additives except a drug active is added to the master batch feed tank 24. The components for pre-mix or master batch 22 are desirably formed in a mixer (not shown) prior to their addition into the master batch feed tank 24. Then a pre-determined amount of the master batch is controllably fed via a first metering pump 26 and control valve 28 to either or both of the first and second mixers, 30, 30′. The present invention, however, is not limited to the use of two mixers, 30, 30′, and any number of mixers may suitably be used. Moreover, the present invention is not limited to any particular sequencing of the mixers 30, 30′, such as parallel sequencing as depicted in FIG. 6, and other sequencing or arrangements of mixers, such as series or combination of parallel and series, may suitably be used. The required amount of the drug or other ingredient, such as a flavor, is added to the desired mixer through an opening, 32, 32′, in each of the mixers, 30, 30′. Desirably, the residence time of the pre-mix or master batch 22 is minimized in the mixers 30, 30′. While complete dispersion of the drug into the pre-mix or master batch 22 is desirable, excessive residence times may result in leaching or dissolving of the drug, especially in the case for a soluble drug. Thus, the mixers 30, 30′ are often smaller, i.e. lower residence times, as compared to the primary mixers (not shown) used in forming the pre-mix or master batch 22. After the drug has been blended with the master batch pre-mix for a sufficient time to provide a uniform matrix, a specific amount of the uniform matrix is then fed to the pan 36 through the second metering pumps, 34, 34′. The metering roller 38 determines the thickness of the film 42 and applies it to the application roller. The film 42 is finally formed on the substrate 44 and carried away via the support roller 46.

While the proper viscosity uniformity in mixture and stable suspension of particles, and casting method are important in the initial steps of forming the composition and film to promote uniformity, the method of drying the wet film is also important. Although these parameters and properties assist uniformity initially, a controlled rapid drying process ensures that the uniformity will be maintained until the film is dry.

The wet film is then dried using controlled bottom drying or controlled microwave drying, desirably in the absence of external air currents or heat on the top (exposed) surface of the film 48 as described herein. Controlled bottom drying or controlled microwave drying advantageously allows for vapor release from the film without the disadvantages of the prior art. Conventional convection air drying from the top is not employed because it initiates drying at the top uppermost portion of the film, thereby forming a barrier against fluid flow, such as the evaporative vapors, and thermal flow, such as the thermal energy for drying. Such dried upper portions serve as a barrier to further vapor release as the portions beneath are dried, which results in non-uniform films. As previously mentioned some top air flow can be used to aid the drying of the films of the present invention, but it must not create a condition that would cause particle movement or a rippling effect in the film, both of which would result in non-uniformity. If top air is employed, it is balanced with the bottom air drying to avoid non-uniformity and prevent film lift-up on the carrier belt. A balance top and bottom air flow may be suitable where the bottom air flow functions as the major source of drying and the top air flow is the minor source of drying. The advantage of some top air flow is to move the exiting vapors away from the film thereby aiding in the overall drying process. The use of any top air flow or top drying, however, must be balanced by a number of factors including, but not limited, to rheological properties of the composition and mechanical aspects of the processing. Any top fluid flow, such as air, also must not overcome the inherent viscosity of the film-forming composition. In other words, the top air flow cannot break, distort or otherwise physically disturb the surface of the composition. Moreover, air velocities are desirably below the yield values of the film, i.e., below any force level that can move the liquids in the film-forming compositions. For thin or low viscosity compositions, low air velocity must be used. For thick or high viscosity compositions, higher air velocities may be used. Furthermore, air velocities are desirable low so as to avoid any lifting or other movement of the film formed from the compositions.

Moreover, the films of the present invention may contain particles that are sensitive to temperature, such as flavors, which may be volatile, or drugs, proteins, or antigens, which may have a low degradation temperature. In such cases, the drying temperature may be decreased while increasing the drying time to adequately dry the uniform films of the present invention. Furthermore, bottom drying also tends to result in a lower internal film temperature as compared to top drying. In bottom drying, the evaporating vapors more readily carry heat away from the film as compared to top drying which lowers the internal film temperature. Such lower internal film temperatures often result in decreased drug degradation and decreased loss of certain volatiles, such as flavors.

During film preparation, it may be desirable to dry films at high temperatures. High heat drying produces uniform films, and leads to greater efficiencies in film production. Films containing sensitive active components, however, may face degradation problems at high temperatures. Degradation is the “decomposition of a compound . . . exhibiting well-defined intermediate products.” The American Heritage Dictionary of the English Language (4^{th} ed. 2000). Degradation of an active component is typically undesirable as it may cause instability, inactivity, and/or decreased potency of the active component. For instance, if the active component is a drug or bioactive material, this may adversely affect the safety or efficacy of the final pharmaceutical product. Additionally, highly volatile materials will tend to be quickly released from this film upon exposure to conventional drying methods.

Degradation of an active component may occur through a variety of processes, such as, hydrolysis, oxidation, and light degradation, depending upon the particular active component. Moreover, temperature has a significant effect on the rate of such reactions. The rate of degradation typically doubles for every 10° C. increase in temperature. Therefore, it is commonly understood that exposing an active component to high temperatures will initiate and/or accelerate undesirable degradation reactions.

Proteins are one category of useful active ingredients that will degrade, denature, or otherwise become inactive when they are exposed to high temperatures for extended periods of time. Proteins serve a variety of functions in the body such as enzymes, structural elements, hormones and immunoglobulins. Examples of proteins include enzymes such as pancreatin, trypsin, pancrelipase, chymotrypsin, hyaluronidase, sutilains, streptokinaw, urokinase, altiplase, papain, bromelainsdiastase, structural elements such as collagen and albumin, hormones such as thyroliberin, gonadoliberin, adrenocorticottropin, corticotrophin, cosyntropin, sometrem, somatropion, prolactin, thyrotropin, somatostatin, vasopressin, felypressin, lypressin, insulin, glucagons, gastrin, pentagastrin, secretin, cholecystokinin-pancreozymin, and immunomodulators which may include polysaccharides in addition to glycoproteins including cytokines which are useful for the inhibition and prevention of malignant cell growth such as tumor growth. A suitable method for the production of some useful glycoproteins is disclosed in U.S. Pat. No. 6,281,337 to Cannon-Carlson, et al., which in incorporated herein in its entirety.

Temperatures that approach 100° C. will generally cause degradation of proteins as well as nucleic acids. For example some glycoproteins will degrade if exposed to a temperature of 70° C. for thirty minutes. Proteins from bovine extract are also known to degrade at such low temperatures. DNA also begins to denature at this temperature.

Applicants have discovered, however, that the films of the present invention may be exposed to high temperatures during the drying process without concern for degradation, loss of activity or excessive evaporation due to the inventive process for film preparation and forming. In particular, the films may be exposed to temperatures that would typically lead to degradation, denaturization, or inactivity of the active component, without causing such problems. According to the present invention, the manner of drying may be controlled to prevent deleterious levels of heat from reaching the active component.

As discussed herein, the flowable mixture is prepared to be uniform in content in accordance with the teachings of the present invention. Uniformity must be maintained as the flowable mass was formed into a film and dried. During the drying process of the present invention, several factors produce uniformity within the film while maintaining the active component at a safe temperature, i.e., below its degradation temperature. First, the films of the present invention have an extremely short heat history, usually only on the order of minutes, so that total temperature exposure is minimized to the extent possible. The films are controllably dried to prevent aggregation and migration of components, as well as preventing heat build up within. Desirably, the films are dried from the bottom. Controlled bottom drying, as described herein, prevents the formation of a polymer film, or skin, on the top surface of the film. As heat is conducted from the film bottom upward, liquid carrier, e.g., water, rises to the film surface. The absence of a surface skin permits rapid evaporation of the liquid carrier as the temperature increases, and thus, concurrent evaporative cooling of the film. Due to the short heat exposure and evaporative cooling, the film components such as drag or volatile actives remain unaffected by high temperatures. In contrast, skinning on the top surface traps liquid carrier molecules of increased energy within the film, thereby causing the temperature within the film to rise and exposing active components to high, potentially deleterious temperatures.

Second, thermal mixing occurs within the film due to bottom heating and absence of surface skinning. Thermal mixing occurs via convection currents in the film. As heat is applied to the bottom of the film, the liquid near the bottom increases in temperature, expands, and becomes less dense. As such, this hotter liquid rises and cooler liquid takes its place. While rising, the hotter liquid mixes with the cooler liquid and shares thermal energy with it, i.e., transfers heat. As the cycle repeats, thermal energy is spread throughout the film.

Robust thermal mixing achieved by the controlled drying process of the present invention produces uniform heat diffusion throughout the film. In the absence of such thermal mixing, “hot spots” may develop. Pockets of heat in the film result in the formation of particle aggregates or danger areas within the film and subsequent non-uniformity. The formation of such aggregates or agglomerations is undesirable because it leads to non-uniform films in which the active may be randomly distributed. Such uneven distribution may lead to large differences in the amount of active per film, which is problematic from a safety and efficacy perspective.

Furthermore, thermal mixing helps to maintain a lower overall temperature inside the film. Although the film surfaces may be exposed to a temperature above that at which the active component degrades, the film interior may not reach this temperature. Due to this temperature differential, the active does not degrade.

For instance, the films of the present invention desirably are dried for 10 minutes or less. Drying the films at 80° C. for 10 minutes produces a temperature differential of about 5° C. This means that after 10 minutes of drying, the temperature of the inside of the film is 5° C. less than the outside exposure temperature. In many cases, however, drying times of less than 10 minutes are sufficient, such as 4 to 6 minutes. Drying for 4 minutes may be accompanied by a temperature differential of about 30° C., and drying for 6 minutes may be accompanied by a differential of about 25° C. Due to such large temperature differentials, the films may be dried at efficient, high temperatures without causing heat sensitive actives to degrade.

FIG. 8 is a sequential representation of the drying process of the present invention. After mechanical mixing, the film may be placed on a conveyor for continued thermal mixing during the drying process. At the outset of the drying process, depicted in Section A, the film 1 preferably is heated from the bottom 10 as it is travels via conveyor (not shown). Heat may be supplied to the film by a heating mechanism, such as, but not limited to, the dryer depicted in FIG. 7. As the film is heated, the liquid carrier, or volatile (“V”), begins to evaporate, as shown by upward arrow 50. Thermal mixing also initiates as hotter liquid, depicted by arrow 30, rises and cooler liquid, depicted by arrow 40, takes its place. Because no skin forms on the top surface 20 of the film 1, as shown in Section B the volatile liquid continues to evaporate 50 and thermal mixing 30/40 continues to distribute thermal energy throughout the film. Once a sufficient amount of the volatile liquid has evaporated, thermal mixing has produced uniform heat diffusion throughout the film 1. The resulting dried film 1 is a visco-elastic solid, as depicted in Section C. The components desirably are locked into a uniform distribution throughout the film. Although minor amounts of liquid carrier, i.e., water, may remain subsequent to formation of the visco-elastic, the film may be dried further without movement of the particles, if desired.

Furthermore, particles or particulates may be added to the film-forming composition or matrix after the composition or matrix is cast into a film. For example, particles may be added to the film 42 prior to the drying of the film 42. Particles may be controllably metered to the film and disposed onto the film through a suitable technique, such as through the use of a doctor blade (not shown) which is a device which marginally or softly touches the surface of the film and controllably disposes the particles onto the film surface. Other suitable, but non-limiting, techniques include the use of an additional roller to place the particles on the film surface, spraying the particles onto the film surface, and the like. The particles may be placed on either or both of the opposed film surfaces, i.e., the top and/or bottom film surfaces. Desirably, the particles are securably disposed onto the film, such as being embedded into the film. Moreover, such particles are desirably not fully encased or fully embedded into the film, but remain exposed to the surface of the film, such as in the case where the particles are partially embedded or partially encased.

The particles may be any useful organoleptic agent, cosmetic agent, pharmaceutical agent, or combinations thereof. Desirably, the pharmaceutical agent is a taste-masked or a controlled-release pharmaceutical agent. Useful organoleptic agents include flavors and sweeteners. Useful cosmetic agents include breath freshening or decongestant agents, such as menthol, including menthol crystals.

Although the inventive process is not limited to any particular apparatus for the above-described desirable drying, one particular useful drying apparatus 50 is depicted in FIG. 7. Drying apparatus 50 is a nozzle arrangement for directing hot fluid, such as but not limited to hot air, towards the bottom of the film 42 which is disposed on substrate 44. Hot air enters the entrance end 52 of the drying apparatus and travels vertically upward, as depicted by vectors 54, towards air deflector 56. The air deflector 56 redirects the air movement to minimize upward force on the film 42. As depicted in FIG. 7, the air is tangentially directed, as indicated by vectors 60 and 60′, as the air passes by air deflector 56 and enters and travels through chamber portions 58 and 58′ of the drying apparatus 50. With the hot air flow being substantially tangential to the film 42, lifting of the film as it is being dried is thereby minimized. While the air deflector 56 is depicted as a roller, other devices and geometries for deflecting air or hot fluid may suitable be used. Furthermore, the exit ends 62 and 62′ of the drying apparatus 50 are flared downwardly. Such downward flaring provides a downward force or downward velocity vector, as indicated by vectors 64 and 64′, which tend to provide a pulling or drag effect of the film 42 to prevent lifting of the film 42. Lifting of the film 42 may not only result in non-uniformity in the film or otherwise, but may also result in non-controlled processing of the film 42 as the film 42 and/or substrate 44 lift away from the processing equipment.

Monitoring and control of the thickness of the film also contributes to the production of a uniform film by providing a film of uniform thickness. The thickness of the film may be monitored with gauges such as Beta Gauges. A gauge may be coupled to another gauge at the end of the drying apparatus, i.e. drying oven or tunnel, to communicate through feedback loops to control and adjust the opening in the coating apparatus, resulting in control of uniform film thickness.

The film products are generally formed by combining a properly selected polymer and polar solvent, as well as any active ingredient or filler as desired. Desirably, the solvent content of the combination is at least about 30% by weight of the total combination. The matrix formed by this combination is formed into a film, desirably by roll coating, and then dried, desirably by a rapid and controlled drying process to maintain the uniformity of the film, more specifically, a non-self-aggregating uniform heterogeneity. The resulting film will desirably contain less than about 10% by weight solvent, more desirably less than about 8% by weight solvent, even more desirably less than about 6% by weight solvent and most desirably less than about 2%. The solvent may be water, a polar organic solvent including, but not limited to, ethanol, isopropanol, acetone, methylene chloride, or any combination thereof.

In alternative embodiments, the film products of the present invention may be formed by extrusion rather than casting methods. Extrusion is particularly useful for film compositions containing polyethylene oxide-based polymer components, as discussed below. For instance, a single screw extrusion process may be employed in accordance with the present invention. According to such an extrusion process, pressure builds in the polymer melt so that it may be extruded through a die or injected into a mold.

As further explanation, a single screw extruder for use in the process of the present invention may include a barrel 300 containing a number of zones 200, as shown in the extruder 100 depicted in FIG. 37. These zones 200 may have varying temperatures and pressures. For instance, it may be desirable for the zones to increase in temperature as the composition proceeds through the barrel 300 to the extrusion die 400. Any number of zones may be included in accordance with the present invention. In addition, the speed of extrusion may be controlled to produce desired film properties. For example, the extrusion composition may be held for an extended time period in the screw mixing chamber. Although this discussion is directed to single screw extrusion, other forms of extrusion are known to those skilled in the art and are considered well within the scope of the present invention.

Consideration of the above discussed parameters, such as but not limited to rheology properties, viscosity, mixing method, casting method and drying method, also impact material selection for the different components of the present invention. Furthermore, such consideration with proper material selection and controlled drying provides the compositions of the present invention, including a pharmaceutical and/or cosmetic dosage form or film product having a uniformity of content in the amount pharmaceutical and/or cosmetic active in an individual dosage unit as sampled from the film such that the amount of active sampled varies no more than about 10% or less percent difference; and desirably varies no more than about 9% or less percent difference, more desirably varies no more than about 8% or less percent difference, even more desirably varies no more than about 7% or less percent difference, even more desirably varies no more than about 6% or less percent difference, even more desirably varies no more than about 5% or less percent difference, even more desirably varies no more than about 4% or less percent difference, even more desirably varies no more than about 3% or less percent difference, even more desirably varies no more than about 2% or less percent difference, even more desirably varies no more than about 1% or less percent difference, even more desirably varies no more than about 0.5% or less percent difference.

The percent difference in active can be calculated as follows. The amount of active (A_{N(i)}) actually present in each sampled individual dosage unit from the same lot (N) as determined by taking the difference between the amount of active in the sample with the most active (Max_{LOT(N)}) minus the amount of active in the sample with the least amount of active (Min_{LOT(N)}) and dividing the difference by the average amount of active in the lot samples (Lot_{(N)} Sample Average). That is: (Max_{LOT(N)}−Min_{LOT(N)})/((A_{N(1)+}A_{N(2)+++}A_{N(10)})/10).

Still furthermore, such consideration with proper material selection and controlled drying provides the compositions of the present invention, including a pharmaceutical and/or cosmetic dosage form or film product having a uniformity of content in the amount pharmaceutical and/or cosmetic active in an individual dosage unit as sampled across different lots of film is determined through establishing the amount of active actually present in each sampled individual dosage unit from the films and comparing that amount of active with a “desired”, or “target” or “labeled” amount of active contained therein. The target amount of active, when it is a pharmaceutical, is referred to as the “Label Claim”, thus identifying the amount of pharmaceutical and/or cosmetic active in the film to a user. The desired amount is 100% of the target amount. Each individual dosage unit film sampled from any individual lot must have the desired content of pharmaceutical active, such that the amount of active varies no more than about 10% or less from the desired or target or labeled amount; and desirably varies no more than about 9% or less, more desirably varies no more than about 8% or less, even more desirably varies no more than about 7% or less, even more desirably varies no more than about 6% or less, even more desirably varies no more than about 5% or less, even more desirably varies no more than about 4% or less, even more desirably varies no more than about 3% or less, even more desirably varies no more than about 2% or less, even more desirably varies no more than about 1% or less, even more desirably varies no more than about 0.5% or less, all of these cascading amounts again from a desired or target or labeled amount.

### Film-Forming Polymers

The polymer may be water soluble, water swellable, water insoluble, or a combination of one or more either water soluble, water swellable or water insoluble polymers. The polymer may include cellulose or a cellulose derivative. Specific examples of useful water soluble polymers include, but are not limited to, polyethylene oxide (PEO), pullulan, hydroxypropylmethyl cellulose (HPMC), hydroxyethyl cellulose (HPC), hydroxypropyl cellulose, polyvinyl pyrrolidone, carboxymethyl cellulose, polyvinyl alcohol, sodium aginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water insoluble polymers include, but are not limited to, ethyl cellulose, hydroxypropyl ethyl cellulose, cellulose acetate phthalate, hydroxypropyl methyl cellulose phthalate and combinations thereof.

As used herein the phrase “water soluble polymer” and variants thereof refer to a polymer that is at least partially soluble in water, and desirably fully or predominantly soluble in water, or absorbs water. Polymers that absorb water are often referred to as being water swellable polymers. The materials useful with the present invention may be water soluble or water swellable at room temperature and other temperatures, such as temperatures exceeding room temperature. Moreover, the materials may be water soluble or water swellable at pressures less than atmospheric pressure. Desirably, the water soluble polymers are water soluble or water swellable having at least 20 percent by weight water uptake. Water swellable polymers having a 25 or greater percent by weight water uptake are also useful. Films or dosage forms of the present invention formed from such water soluble polymers are desirably sufficiently water soluble to be dissolvable upon contact with bodily fluids.

Other polymers useful for incorporation into the films of the present invention include biodegradable polymers, copolymers, block polymers and combinations thereof. Among the known useful polymers or polymer classes which meet the above criteria are: poly(glycolic acid) (PGA), poly(lactic acid) (PLA), polydioxanoes, polyoxalates, poly(α-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of a-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100 L, believed to be 100% lactide having a melting point within the range of 338°-347° F. (170°-175° C.); lactide/glycolide 100 L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.).

The Biodel materials represent a family of various polyanhydrides which differ chemically.

Although a variety of different polymers may be used, it is desired to select polymers to provide a desired viscosity of the mixture prior to drying. For example, if the active or other components are not soluble in the selected solvent, a polymer that will provide a greater viscosity is desired to assist in maintaining uniformity. On the other hand, if the components are soluble in the solvent, a polymer that provides a lower viscosity may be preferred.

The polymer plays an important role in affecting the viscosity of the film. Viscosity is one property of a liquid that controls the stability of the active in an emulsion, a colloid or a suspension. Generally the viscosity of the matrix will vary from about 400 cps to about 100,000 cps, preferably from about 800 cps to about 60,000 cps, preferably from about 400 cps to about 5,000 cps, preferably from about 5,000 cps to about 10,000 cps, preferably from about 10,000 cps to about 20,000 cps, preferably from about 20,000 cps to about 30,000 cps, preferably from about 30,000 cps to about 40,000 cps, preferably from about 40,000 cps to about 50,000 cps, preferably from about 50,000 cps to about 60,000 cps, preferably from about 60,000 cps to about 70,000 cps, preferably from about 80,000 cps to about 90,000 cps, preferably from about 90,000 cps to about 100,000 cps, and most preferably from about 1,000 cps to about 40,000 cps. Desirably, the viscosity of the film-forming matrix will rapidly increase upon initiation of the drying process.

In some embodiments relating to certain pharmaceutical and biological actives, applicants have found that generally the viscosity of the matrix may vary from about 7,000 cps to about 30,000 cps, preferably from about 10,000 cps to about 18,000 cps, and most preferably from about 12,000 cps to about 15,000 cps, when using a Brookfield DV-II+ Pro, and a spindle 27 (at 2, 5 and 10 revolutions per minute), and at 25° C. Desirably, the viscosity of the film-forming matrix will rapidly increase upon initiation of the drying process.

The viscosity may be adjusted based on the selected active depending on the other components within the matrix. For example, if the component is not soluble within the selected solvent, a proper viscosity may be selected to prevent the component from settling which would adversely affect the uniformity of the resulting film. The viscosity may be adjusted in different ways. To increase viscosity of the film matrix, the polymer may be chosen of a higher molecular weight or crosslinkers may be added, such as salts of calcium, sodium and potassium. The viscosity may also be adjusted by adjusting the temperature or by adding a viscosity increasing component. Components that will increase the viscosity or stabilize the emulsion/suspension include higher molecular weight polymers and polysaccharides and gums, which include without limitation, alginate, carrageenan, hydroxypropyl methyl cellulose, locust bean gum, guar gum, xanthan gum, dextran, gum arabic, gellan gum and combinations thereof.

It has also been observed that certain polymers which when used alone would ordinarily require a plasticizer to achieve a flexible film, can be combined without a plasticizer and yet achieve flexible films. For example, HPMC and HPC when used in combination provide a flexible, strong film with the appropriate plasticity and elasticity for manufacturing and storage. No additional plasticizer or polyalcohol is needed for flexibility.

Additionally, polyethylene oxide (PEO), when used alone or in combination with a hydrophilic cellulosic polymer, achieves flexible, strong films. Additional plasticizers or polyalcohols are not needed for flexibility. Non-limiting examples of suitable cellulosic polymers for combination with PEO include HPC and HPMC. PEO and HPC have essentially no gelation temperature, while HPMC has a gelation temperature of 58-64° C. (Methocel EF available from Dow Chemical Co.). Moreover, these films are sufficiently flexible even when substantially free of organic solvents, which may be removed without compromising film properties. As such, if there is no solvent present, then there is no plasticizer in the films. PEO based films also exhibit good resistance to tearing, little or no curling, and fast dissolution rates when the polymer component contains appropriate levels of PEO.

To achieve the desired film properties, the level and/or molecular weight of PEO in the polymer component may be varied. Modifying the PEO content affects properties such as tear resistance, dissolution rate, and adhesion tendencies. Thus, one method for controlling film properties is to modify the PEO content. For instance, in some embodiments rapid dissolving films are desirable. By modifying the content of the polymer component, the desired dissolution characteristics can be achieved.

In accordance with the present invention, PEO desirably ranges from about 20% to 100% by weight in the polymer component. In some embodiments, the amount of PEO desirably ranges from about 1 mg to about 200 mg. The hydrophilic cellulosic polymer ranges from about 0% to about 80% by weight, or in a ratio of up to about 4:1 with the PEO, and desirably in a ratio of about 1:1.

In some embodiments, it may be desirable to vary the PEO levels to promote certain film properties. To obtain films with high tear resistance and fast dissolution rates, levels of about 50% or greater of PEO in the polymer component are desirable. To achieve adhesion prevention, i.e., preventing the film from adhering to the roof of the mouth, PEO levels of about 20% to 75% are desirable. In some embodiments, however, adhesion to the roof of the mouth may be desired, such as for administration to animals or children. In such cases, higher levels of PEO may be employed. More specifically, structural integrity and dissolution of the film can be controlled such that the film can adhere to mucosa and be readily removed, or adhere more firmly and be difficult to remove, depending on the intended use.

The molecular weight of the PEO may also be varied. High molecular weight PEO, such as about 4 million, may be desired to increase mucoadhesivity of the film. More desirably, the molecular weight may range from about 100,000 to 900,000, more desirably from about 100,000 to 600,000, and most desirably from about 100,000 to 300,000. In some embodiments, it may be desirable to combine high molecular weight (600,000 to 900,000) with low molecular weight (100,000 to 300,000) PEOs in the polymer component.

For instance, certain film properties, such as fast dissolution rates and high tear resistance, may be attained by combining small amounts of high molecular weight PEOs with larger amounts of lower molecular weight PEOs. Desirably, such compositions contain about 60% or greater levels of the lower molecular weight PEO in the PEO-blend polymer component.

To balance the properties of adhesion prevention, fast dissolution rate, and good tear resistance, desirable film compositions may include about 50% to 75% low molecular weight PEO, optionally combined with a small amount of a higher molecular weight PEO, with the remainder of the polymer component containing a hydrophilic cellulosic polymer (HPC or HPMC).

### Controlled Release Films

The term “controlled release” is intended to mean the release of active at a pre-selected or desired rate. This rate will vary depending upon the application. Desirable rates include fast or immediate release profiles as well as delayed, sustained or sequential release. Combinations of release patterns, such as initial spiked release followed by lower levels of sustained release of active are contemplated. Pulsed drug releases are also contemplated.

The polymers that are chosen for the films of the present invention may also be chosen to allow for controlled disintegration of the active. This may be achieved by providing a substantially water insoluble film that incorporates an active that will be released from the film over time. This may be accomplished by incorporating a variety of different soluble or insoluble polymers and may also include biodegradable polymers in combination. Alternatively, coated controlled release active particles may be incorporated into a readily soluble film matrix to achieve the controlled release property of the active inside the digestive system upon consumption.

Films that provide a controlled release of the active are particularly useful for buccal, gingival, sublingual and vaginal applications. The films of the present invention are particularly useful where mucosal membranes or mucosal fluid is present due to their ability to readily wet and adhere to these areas.

The convenience of administering a single dose of a medication which releases active ingredients in a controlled fashion over an extended period of time as opposed to the administration of a number of single doses at regular intervals has long been recognized in the pharmaceutical arts. The advantage to the patient and clinician in having consistent and uniform blood levels of medication over an extended period of time are likewise recognized. The advantages of a variety of sustained release dosage forms are well known. However, the preparation of a film that provides the controlled release of an active has advantages in addition to those well-known for controlled release tablets. For example, thin films are difficult to inadvertently aspirate and provide an increased patient compliance because they need not be swallowed like a tablet. Moreover, certain embodiments of the inventive films are designed to adhere to the buccal cavity and tongue, where they controllably dissolve. Furthermore, thin films may not be crushed in the manner of controlled release tablets which is a problem leading to abuse of drugs such as Oxycontin.

The actives employed in the present invention may be incorporated into the film compositions of the present invention in a controlled release form. For example, particles of drug may be coated with polymers such as ethyl cellulose or polymethacrylate, commercially available under brand names such as Aquacoat ECD and Eudragit E-100, respectively. Solutions of drug may also be absorbed on such polymer materials and incorporated into the inventive film compositions. Other components such as fats and waxes, as well as sweeteners and/or flavors may also be employed in such controlled release compositions.

The actives may be taste-masked prior to incorporation into the film composition, as set forth in co-pending PCT application titled, Uniform Films For Rapid Dissolve Dosage Form Incorporating Taste-Masking Compositions, (based on U.S. Provisional Application No. Express Mail Label No.: EU552991605 US of the same title, filed Sep. 27, 2003, attorney docket No. 1199-15P) the entire subject matter of which is incorporated by reference herein.

### Actives

When an active is introduced to the film, the amount of active per unit area is determined by the uniform distribution of the film. For example, when the films are cut into individual dosage forms, the amount of the active in the dosage form can be known with a great deal of accuracy. This is achieved because the amount of the active in a given area is substantially identical to the amount of active in an area of the same dimensions in another part of the film. The accuracy in dosage is particularly advantageous when the active is a medicament, i.e. a drug.

Triptants are a class of analgesics that bind to and are agonists of serotonin receptors. The triptans include rizatriptan, sumatriptan, zolmitriptan, naratriptan, almotriptan, eletriptan, and frovatriptan.

The films of the present invention contain rizatriptan or a pharmaceutically acceptable salt thereof. Rizatrptan is a 5-HT₁ agonist having the following structure:

[chemical structure]

Pharmaceutically acceptable salts of rizatriptan include rizatrptan benzoate, rizatrptan oxalate, rizatrptan succinate, rizatrptan citrate, and mixtures thereof,

The rizatriptan may be present in the film products of the present invention in a therapeutically effective amount. A “therapeutically acceptable amount” is an amount of an active ingredient that produces a desired therapeutic response in a subject. In embodiments of the present invention, the rizatrptan is present in an amount from about 1 to about 50 milligrams per dose, preferably, from about 2 to about 20 milligrams, and even more preferably in an amount of about 5 milligrams or about 10 milligrams.

The Applicants have discovered that the film products of the present invention produce a pharmacokinetic profile for the absorption of rizatriptan that is bioequivalent to the commercially available rizatriptan dissolvable tablet product MAXALT®, in terms of Cmax and area under the curve (AUC) for the plasma level of rizatriptan, while providing a faster onset of action, as measured by Tₘₐₓ.

As used herein, the term Cmax refers to the mean maximum plasma concentration after administration of the composition to a human subject. As also used herein, the term AUC refers to the mean area under the plasma concentration-time curve value after administration of the compositions formed herein. As will be set forth in more detail below, the term “optimizing the absorption” does not necessarily refer to reaching the maximum absorption of the composition. The “optimum” absorption may be, for example, a level that provides a bioequivalent absorption as administration of the currently available MAXALT® tablet.

The term “bioequivalent” means obtaining 80% to 125% of the Cmax and AUC values for a given active in a different product. For example, assuming Cmax and AUC₀₋₁₂ values of rizatriptan for a commercially-available MAXALT® tablet (containing 10 mg rizatriptan) are 20.52 ng/ml and 70.89 hr*ng/ml, respectively, a bioequivalent product would have a Cmax of rizatriptan in the range of 16.4-25.65 ng/ml, and an AUC value of rizatriptan of 56.7-88.6 hr*ng/ml.

In one embodiment, the film product is a unit dosage form configured such that when the dosage form is administered to human subjects in a fasted state, the time to reach maximum plasma concentration (Tₘₐₓ) of rizatriptan is about 10 minutes to about 75 minutes after administration of the dosage form, preferably from about 15 to about 45 minutes, and more preferably about 30 minutes or less.

In one embodiment, the film product is a unit dosage form containing about 14.5 milligrams of rizatriptan benzoate and has a mean AUC₀₋₁₂ value of from about 35 to about 150 hr*ng/ml, preferably from about 56.7-88.6 hr*ng/ml, preferably from about 60-80 hr*ng/ml, more preferably about 71 hr*ng/ml. In another embodiment, the film product is a unit dosage form containing about 7.25 milligrams of rizatriptan benzoate and has a mean AUC₀₋₁₂ value of from about 17.75 to about 75 hr*ng/ml, preferably from about 28-45 hr*ng/ml, preferably from about 30-40 hr*ng/ml, more preferably about 35.5 hr*ng/ml.

The films of the present invention may include, without limitation, an additional active component. The additional active components that may be incorporated into the films of the present invention include, without limitation pharmaceutical and cosmetic actives, drugs, medicaments, proteins, antigens or allergens such as ragweed pollen, spores, microorganisms, seeds, mouthwash components, flavors, fragrances, enzymes, preservatives, sweetening agents, colorants, spices, vitamins and combinations thereof.

A wide variety of medicaments, bioactive active substances and pharmaceutical compositions may be included in the dosage forms of the present invention. Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of medicating active ingredients contemplated for use in the present invention include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (available as Oxycontin®), ibuprofen, aspirin, acetaminophen, and combinations thereof that may optionally include caffeine.

Other preferred drugs for other preferred active ingredients for use in the present invention include anti-diarrheals such as immodium AD, anti-histamines, anti-tussives, decongestants, vitamins, and breath fresheners. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film compositions of the present invention.

Also contemplated for use herein are anxiolytics such as alprazolam (available as Xanax®); anti-psychotics such as clozopin (available as Clozaril®) and haloperidol (available as Haldol®); non-steroidal anti-inflammatories (NSAID's) such as dicyclofenacs (available as Voltaren®) and etodolac (available as Lodine®), anti-histamines such as loratadine (available as Claritin®), astemizole (available as Hismanal™), nabumetone (available as Relafen®), and Clemastine (available as Tavist®); anti-emetics such as ondensetron and granisetron hydrochloride (available as Kytril®) and nabilone (available as Cesamet™); bronchodilators such as Bentolin®, albuterol sulfate (available as Proventil®); anti-depressants such as fluoxetine hydrochloride (available as Prozac®), sertraline hydrochloride (available as Zoloft®), and paroxtine hydrochloride (available as Paxil®); anti-migraines such as Imigra®, ACE-inhibitors such as enalaprilat (available as Vasotec®), captopril (available as Capoten®) and lisinopril (available as Zestril®); anti-Alzheimer's agents, such as nicergoline; and Ca^{H}-antagonists such as nifedipine (available as Procardia® and Adalat®), and verapamil hydrochloride (available as Calan®).

Erectile dysfunction therapies include, but are not limited to, drugs for facilitating blood flow to the penis, and for effecting autonomic nervous activities, such as increasing parasympathetic (cholinergic) and decreasing sympathetic (adrenersic) activities. Useful non-limiting drugs include sildenafils, such as Viagra®, tadalafils, such as Clalis®, vardenafils, apomorphines, such as Uprima®, yohimbine hydrochlorides such as Aphrodyne®, and alprostadils such as Caverject®.

The popular H₂-antagonists which are contemplated for use in the present invention include cimetidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono- or di-basic calcium phosphate, tricalcium phosphate, potassium bicarbonate, sodium tartrate, sodium bicarbonate, magnesium aluminosilicates, tartaric acids and salts.

The pharmaceutically active agents employed in the present invention may include allergens or antigens, such as, but not limited to, plant pollens from grasses, trees, or ragweed; animal danders, which are tiny scales shed from the skin and hair of cats and other furred animals; insects, such as house dust mites, bees, and wasps; and drugs, such as penicillin.

An anti-oxidant may also be added to the film to prevent the degradation of an active, especially where the active is photosensitive.

Cosmetic active agents may include breath freshening compounds like menthol, other flavors or fragrances, especially those used for oral hygiene, as well as actives used in dental and oral cleansing such as quaternary ammonium bases. The effect of flavors may be enhanced using flavor enhancers like tartaric acid, citric acid, vanillin, or the like.

Also color additives can be used in preparing the films. Such color additives include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide.

Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors.

The films containing flavorings may be added to provide a hot or cold flavored drink or soup. These flavorings include, without limitation, tea and soup flavorings such as beef and chicken.

Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

When the active is combined with the polymer in the solvent, the type of matrix that is formed depends on the solubilities of the active and the polymer. If the active and/or polymer are soluble in the selected solvent, this may form a solution. However, if the components are not soluble, the matrix may be classified as an emulsion, a colloid, or a suspension.

### Dosages

The film products of the present invention are capable of accommodating a wide range of amounts of the active ingredient. The films are capable of providing an accurate dosage amount (determined by the size of the film and concentration of the active in the original polymer/water combination) regardless of whether the required dosage is high or extremely low. Therefore, depending on the type of active or pharmaceutical composition that is incorporated into the film, the active amount may be as high as about 300 mg, desirably up to about 150 mg or as low as the nanogram range, or any amount therebetween.

The film products and methods of the present invention are well suited for high potency, low dosage drugs. This is accomplished through the high degree of uniformity of the films. Therefore, low dosage drugs, particularly more potent racemic mixtures of actives are desirable.

### Anti-Foaming and De-Foaming Compositions

Anti-foaming and/or de-foaming components may also be used with the films of the present invention. These components aid in the removal of air, such as entrapped air, from the film-forming compositions. As described above, such entrapped air may lead to non-uniform films. Simethicone is one particularly useful anti-foaming and/or de-foaming agent. The present invention, however, is not so limited and other anti-foam and/or de-foaming agents may suitable be used.

As a related matter, simethicone and related agents may be employed for densification purposes. More specifically, such agents may facilitate the removal of voids, air, moisture, and similar undesired components, thereby providing denser, and thus more uniform films. Agents or components which perform this function can be referred to as densification or densifying agents. As described above, entrapped air or undesired components may lead to non-uniform films.

Simethicone is generally used in the medical field as a treatment for gas or colic in babies. Simethicone is a mixture of fully methylated linear siloxane polymers containing repeating units of polydimethylsiloxane which is stabilized with trimethylsiloxy end-blocking unites, and silicon dioxide. It usually contains 90.5-99% polymethylsiloxane and 4-7% silicon dioxide. The mixture is a gray, translucent, viscous fluid which is insoluble in water.

When dispersed in water, simethicone will spread across the surface, forming a thin film of low surface tension. In this way, simethicone reduces the surface tension of bubbles air located in the solution, such as foam bubbles, causing their collapse. The function of simethicone mimics the dual action of oil and alcohol in water. For example, in an oily solution any trapped air bubbles will ascend to the surface and dissipate more quickly and easily, because an oily liquid has a lighter density compared to a water solution. On the other hand, an alcohol/water mixture is known to lower water density as well as lower the water's surface tension. So, any air bubbles trapped inside this mixture solution will also be easily dissipated. Simethicone solution provides both of these advantages. It lowers the surface energy of any air bubbles that trapped inside the aqueous solution, as well as lowering the surface tension of the aqueous solution. As the result of this unique functionality, simethicone has an excellent anti-foaming property that can be used for physiological processes (anti-gas in stomach) as well as any for external processes that require the removal of air bubbles from a product.

In order to prevent the formation of air bubbles in the films of the present invention, the mixing step can be performed under vacuum. However, as soon as the mixing step is completed, and the film solution is returned to the normal atmosphere condition, air will be re-introduced into or contacted with the mixture. In many cases, tiny air bubbles will be again trapped inside this polymeric viscous solution. The incorporation of simethicone into the film-forming composition either substantially reduces or eliminates the formation of air bubbles.

Simethicone may be added to the film-forming mixture as an anti-foaming agent in an amount from about 0.01 weight percent to about 5.0 weight percent, more desirably from about 0.05 weight percent to about 2.5 weight percent, and most desirably from about 0.1 weight percent to about 1.0 weight percent.

### Penetration Enhancers

Penetration enhancers are compounds that increase the rate or efficiency of the penetration of an active through the surface of the body, such as the mucosal surface. Examples of penetration enhancers include sodium lauryl Sulfate, Sodium decyl sulfate, Sodium octyl sulfate, Sodium laureth sulfate, N-Lauryl sarcosinate, Cetyltrimethyl ammonium bromide, Decyltrimethyl ammonium bromide, Benzyldimethyl dodecyl ammonium chloride, Myristyltrimethyl ammonium chloride, Dodecyl pyridinium chloride, Decyldimethyl ammonio propane sulfonate, Myristyldimethyl ammonlo propane sulfonate, Palmityldimethyl ammonio propane sulfonate, ChemBetaine CAS, ChemBctainc Oleyl, Palmltoyl camitine chloride, Nonylphenoxypolyoxyethylene, Polyoxyethylene sorbitan monolaurate, Polyoxyethylene sorbitan monopalmitate, Sorbitan monooleate, Triton-X 100, Sodium deoxycholate, Sodium glycocholate, Cholic acid, Hexanoic acid, Heptanoic acid, Methyllaurate, Isopropyl myrlstate, Isopropyl palmitate, Methyl palmitate, Diethyl sebaccate, Sodium oleate, Urea, Lauryl amine, Caprolactam, Methyl pyrrolidone, Octyl pyrrolidone, Methyl piperazine, Phenyl piperazine, Sodium salicylate, Carbopol934P, Glyccyrhetinic acid, Bromelain, Pinene oxide, Limonene, Cineole, Octyl dodecanol, Fenchone, Menthane, Trimethoxy propylene methyl benzene, and combinations thereof.

### Optional Components

A variety of other components and fillers may also be added to the films of the present invention. These may include, without limitation, surfactants; plasticizers which assist in compatibilizing the components within the mixture; polyalcohols; anti-foaming agents, such as silicone-containing compounds, which promote a smoother film surface by releasing oxygen from the film; thermo-setting gels such as pectin, carageenan, and gelatin, which help in maintaining the dispersion of components; and inclusion compounds, such as cyclodextrins and caged molecules, which improve the solubility and/or stability of certain active components.

The variety of additives that can be incorporated into the inventive compositions may provide a variety of different functions. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active ingredient(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water soluble chemical derivatives of starch, cationically modified acrylates and methacrylates possessing, for example, a tertiary or quaternary amino group, such as the diethylaminoethyl group, which may be quaternized if desired; and other similar polymers.

Such extenders may optionally be added in any desired amount desirably within the range of up to about 80%, desirably about 3% to 50% and more desirably within the range of 3% to 20% based on the weight of all components.

Further additives may be inorganic fillers, such as the oxides of magnesium aluminum, silicon, titanium, etc. desirably in a concentration range of about 0.02% to about 3% by weight and desirably about 0.02% to about 1% based on the weight of all components.

Further examples of additives are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like, added in concentrations ranging from about 0.5% to about 30%, and desirably ranging from about 0.5% to about 20% based on the weight of the polymer.

There may further be added compounds to improve the flow properties of the starch material such as animal or vegetable fats, desirably in their hydrogenated form, especially those which are solid at room temperature. These fats desirably have a melting point of 50° C. or higher. Preferred are tri-glycerides with C₁₂-, C₁₄-, C₁₆-, C₁₈-, C₂₀- and C₂₂-fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e. with C₁₂-, C₁₄-, C₁₆-, C₁₈-, C₂₀- and C₂₂-fatty acids.

The total amounts used of the fats, mono-, di-glycerides and/or lecithins are up to about 5% and preferably within the range of about 0.5% to about 2% by weight of the total composition

It is further useful to add silicon dioxide, calcium silicate, or titanium dioxide in a concentration of about 0.02% to about 1% by weight of the total composition. These compounds act as texturizing agents.

These additives are to be used in amounts sufficient to achieve their intended purpose. Generally, the combination of certain of these additives will alter the overall release profile of the active ingredient and can be used to modify, i.e. impede or accelerate the release.

Lecithin is one surface active agent for use in the present invention. Lecithin can be included in the feedstock in an amount of from about 0.25% to about 2.00% by weight. Other surface active agents, i.e. surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL which is commercially available from BASF, are also useful. Carbowax™ is yet another modifier which is very useful in the present invention. Tweens™ or combinations of surface active agents may be used to achieve the desired hydrophilic-lipophilic balance (“HLB”). The present invention, however, does not require the use of a surfactant and films or film-forming compositions of the present invention may be essentially free of a surfactant while still providing the desirable uniformity features of the present invention.

As additional modifiers which enhance the procedure and product of the present invention are identified, Applicants intend to include all such additional modifiers within the scope of the invention claimed herein.

Other ingredients include binders which contribute to the ease of formation and general quality of the films. Non-limiting examples of binders include starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols.

Further potential additives include solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable actives may fit within the hydrophobic cavity, thereby producing an inclusion complex, which is soluble in water. Accordingly, the formation of the inclusion complex permits very insoluble and/or instable actives to be dissolved in water. A particularly desirable example of such agents are cyclodextrins, which are cyclic carbohydrates derived from starch. Other similar substances, however, are considered well within the scope of the present invention.

### Forming the Film

The films of the present invention must be formed into a sheet prior to drying. After the desired components are combined to form a multi-component matrix, including the polymer, water, and an active or other components as desired, the combination is formed into a sheet or film, by any method known in the art such as extrusion, coating, spreading, casting or drawing the multi-component matrix. If a multi-layered film is desired, this may be accomplished by co-extruding more than one combination of components which may be of the same or different composition. A multi-layered film may also be achieved by coating, spreading, or casting a combination onto an already formed film layer.

Although a variety of different film-forming techniques may be used, it is desirable to select a method that will provide a flexible film, such as reverse roll coating. The flexibility of the film allows for the sheets of film to be rolled and transported for storage or prior to being cut into individual dosage forms. Desirably, the films will also be self-supporting or in other words able to maintain their integrity and structure in the absence of a separate support. Furthermore, the films of the present invention may be selected of materials that are edible or ingestible.

Coating or casting methods are particularly useful for the purpose of forming the films of the present invention. Specific examples include reverse roll coating, gravure coating, immersion or dip coating, metering rod or meyer bar coating, slot die or extrusion coating, gap or knife over roll coating, air knife coating, curtain coating, or combinations thereof, especially when a multi-layered film is desired.

Roll coating, or more specifically reverse roll coating, is particularly desired when forming films in accordance with the present invention. This procedure provides excellent control and uniformity of the resulting films, which is desired in the present invention. In this procedure, the coating material is measured onto the applicator roller by the precision setting of the gap between the upper metering roller and the application roller below it. The coating is transferred from the application roller to the substrate as it passes around the support roller adjacent to the application roller. Both three roll and four roll processes are common.

The gravure coating process relies on an engraved roller running in a coating bath, which fills the engraved dots or lines of the roller with the coating material. The excess coating on the roller is wiped off by a doctor blade and the coating is then deposited onto the substrate as it passes between the engraved roller and a pressure roller.

Offset Gravure is common, where the coating is deposited on an intermediate roller before transfer to the substrate.

In the simple process of immersion or dip coating, the substrate is dipped into a bath of the coating, which is normally of a low viscosity to enable the coating to run back into the bath as the substrate emerges.

In the metering rod coating process, an excess of the coating is deposited onto the substrate as it passes over the bath roller. The wire-wound metering rod, sometimes known as a Meyer Bar, allows the desired quantity of the coating to remain on the substrate. The quantity is determined by the diameter of the wire used on the rod.

In the slot die process, the coating is squeezed out by gravity or under pressure through a slot and onto the substrate. If the coating is 100% solids, the process is termed “Extrusion” and in this case, the line speed is frequently much faster than the speed of the extrusion. This enables coatings to be considerably thinner than the width of the slot.

It may be particularly desirable to employ extrusion methods for forming film compositions containing PEO polymer components. These compositions contain PEO or PEO blends in the polymer component, and may be essentially free of added plasticizers, and/or surfactants, and polyalcohols. The compositions may be extruded as a sheet at processing temperatures of less than about 90° C. Extrusion may proceed by squeezing the film composition through rollers or a die to obtain a uniform matrix. The extruded film composition then is cooled by any mechanism known to those of ordinary skill in the art. For example, chill rollers, air cooling beds, or water cooling beds may be employed. The cooling step is particularly desirable for these film compositions because PEO tends to hold heat.

The gap or knife over roll process relies on a coating being applied to the substrate which then passes through a “gap” between a “knife” and a support roller. As the coating and substrate pass through, the excess is scraped off.

Air knife coating is where the coating is applied to the substrate and the excess is “blown off” by a powerful jet from the air knife. This procedure is useful for aqueous coatings.

In the curtain coating process, a bath with a slot in the base allows a continuous curtain of the coating to fall into the gap between two conveyors. The object to be coated is passed along the conveyor at a controlled speed and so receives the coating on its upper face.

### Drying the Film

The drying step is also a contributing factor with regard to maintaining the uniformity of the film composition. A controlled drying process is particularly important when, in the absence of a viscosity increasing composition or a composition in which the viscosity is controlled, for example by the selection of the polymer, the components within the film may have an increased tendency to aggregate or conglomerate. An alternative method of forming a film with an accurate dosage, that would not necessitate the controlled drying process, would be to cast the films on a predetermined well. With this method, although the components may aggregate, this will not result in the migration of the active to an adjacent dosage form, since each well may define the dosage unit per se.

When a controlled or rapid drying process is desired, this may be through a variety of methods. A variety of methods may be used including those that require the application of heat. The liquid carriers are removed from the film in a manner such that the uniformity, or more specifically, the non-self-aggregating uniform heterogeneity, that is obtained in the wet film is maintained.

Desirably, the film is dried from the bottom of the film to the top of the film. Desirably, substantially no air flow is present across the top of the film during its initial setting period, during which a solid, visco-elastic structure is formed. This can take place within the first few minutes, e.g. about the first 0.5 to about 8.0 minutes of the drying process. Controlling the drying in this manner, prevents the destruction and reformation of the film's top surface, which results from conventional drying methods. This is accomplished by forming the film and placing it on the top side of a surface having top and bottom sides. Then, heat is initially applied to the bottom side of the film to provide the necessary energy to evaporate or otherwise remove the liquid carrier. The films dried in this manner dry more quickly and evenly as compared to air-dried films, or those dried by conventional drying means. In contrast to an air-dried film that dries first at the top and edges, the films dried by applying heat to the bottom dry simultaneously at the center as well as at the edges. This also prevents settling of ingredients that occurs with films dried by conventional means.

The temperature at which the films are dried is about 140° C. or less desirably about 100° C. or less, more desirably about 90° C. or less, and most desirably about 80° C. or less.

The temperature of the film matrix must be maintained below 100° C.

Another method of controlling the drying process, which may be used alone or in combination with other controlled methods as disclosed above includes controlling and modifying the humidity within the drying apparatus where the film is being dried. In this manner, the premature drying of the top surface of the film is avoided.

Additionally, it has also been discovered that the length of drying time can be properly controlled, i.e. balanced with the heat sensitivity and volatility of the components, and particularly the flavor oils and drugs. The amount of energy, temperature and length and speed of the conveyor can be balanced to accommodate such actives and to minimize loss, degradation or ineffectiveness in the final film.

A specific example of an appropriate drying method is that disclosed by Magoon. Magoon is specifically directed toward a method of drying fruit pulp. However, the present inventors have adapted this process toward the preparation of thin films.

The method and apparatus of Magoon are based on an interesting property of water. Although water transmits energy by conduction and convection both within and to its surroundings, water only radiates energy within and to water. Therefore, the apparatus of Magoon includes a surface onto which the fruit pulp is placed that is transparent to infrared radiation. The underside of the surface is in contact with a temperature controlled water bath. The water bath temperature is desirably controlled at a temperature slightly below the boiling temperature of water. When the wet fruit pulp is placed on the surface of the apparatus, this creates a “refractance window.” This means that infrared energy is permitted to radiate through the surface only to the area on the surface occupied by the fruit pulp, and only until the fruit pulp is dry. The apparatus of Magoon provides the films of the present invention with an efficient drying time reducing the instance of aggregation of the components of the film.

Another method of controlling the drying process involves a zone drying procedure. A zone drying apparatus may include a continuous belt drying tunnel having one or more drying zones located within. The conditions of each drying zone may vary, for example, temperature and humidity may be selectively chosen. It may be desirable to sequentially order the zones to provide a stepped up drying effect.

The speed of the zone drying conveyor desirably is continuous. Alternatively, the speed may be altered at a particular stage of the drying procedure to increase or decrease exposure of the film to the conditions of the desired zone. Whether continuous or modified, the zone drying dries the film without surface skinning.

According to an embodiment of the zone drying apparatus 100, shown in FIG. 35, the film 110 may be fed onto the continuous belt 120, which carries the film through the different drying zones. The first drying zone that the film travels through 101 may be a warm and humid zone. The second zone 102 may be hotter and drier, and the third zone 103 may also be hot and dry. These different zones may be continuous, or alternatively, they may be separated, as depicted by the zone drying apparatus 200 in FIG. 36. The zone drying apparatus, in accordance with the present invention, is not limited to three drying zones. The film may travel through lesser or additional drying zones of varying heat and humidity levels, if desired, to produce the controlled drying effect of the present invention.

To further control temperature and humidity, the drying zones may include additional atmospheric conditions, such as inert gases. The zone drying apparatus further may be adapted to include additional processes during the zone drying procedure, such as, for example, spraying and laminating processes, so long as controlled drying is maintained in accordance with the invention.

The films may initially have a wet thickness of about 250 μm to about 3,000 μm, or about 10 mils to about 120 mils, and when dried have a thickness from about 1.5 μm to about 500 μm, or about 0.05 mils to about 10 mils. Desirably, the dried films will have a thickness of about 2 mils to about 8 mils, and more desirably, from about 3 mils to about 6 mils.

### Testing Films for Uniformity of Content

It may be desirable to test the films of the present invention for chemical and physical uniformity during the film manufacturing process. Physical tests may be preferentially used to warn of potential non-uniformity in the production process, so adjustments may be made, while analytical chemical tests and other assay tests may be used to determine uniformity of content in the actual amount of the relevant active in the finished film. Analytical chemical tests and other assay tests may be used at any time throughout the production process as well. In particular, samples of the film may be removed and tested for uniformity of content in film components between various samples. Film thickness and over all appearance may also be checked for uniformity. However, uniformity of content in the amount of active as determined by analytical chemical testing and other assay testing is essential for films containing pharmaceutical and/or biological active components for safety and efficacy reasons.

A method for testing uniformity in accordance with the present invention includes conveying a film through a manufacturing process. This process may include subjecting the film to drying processes, dividing the film into individual dosage units, and/or packaging the dosages, among others. As the film is conveyed through the manufacturing process, for example on a conveyor belt apparatus, it is cut widthwise into at least one portion. The at least one portion has opposing ends that are separate from any other film portion. For instance, if the film is a roll, it may be cut into separate sub-rolls. Cutting the film may be accomplished by a variety of methods, such as with a knife, razor, laser, or any other suitable means for cutting a film.

The cut film then may be sampled by removing small pieces from each of the opposed ends of the portion(s), without disrupting the middle of the portion(s). Leaving the middle section intact permits the predominant portion of the film to proceed through the manufacturing process without interrupting the conformity of the film and creating sample-inducted gaps in the film. Accordingly, the concern of missing doses is alleviated as the film is further processed, e.g., packaged. Moreover, maintaining the completeness of cut portions or sub-rolls throughout the process will help to alleviate the possibility of interruptions in further film processing or packaging due to guilty control issues, for example, alarm stoppage due to notice of missing pieces.

After the end pieces, or sampling sections, are removed from the film portion(s), they may be tested for uniformity in the content of components between samples. Any conventional means for examining and testing the film pieces may be employed, such as, for example, visual inspection, use of analytical equipment, and any other suitable means known to those skilled in the art. If the testing results show non-uniformity between film samples, the manufacturing process may be altered. This can save time and expense because the process may be altered prior to completing an entire manufacturing run. For example, the drying conditions, mixing conditions, compositional components and/or film viscosity may be changed. Altering the drying conditions may involve changing the temperature, drying time, moisture level, and dryer positioning, among others.

Testing of the finished film product additionally involves analytical chemical testing such as High Performance Liquid Chromatography (HPLC) and/or other methods approved for use with the particular finished film product. For example, in the case of a drug and related products such tests must include assaying for the amount of active in accordance with the relevant regulatory requirements, such as those provided by the FDA in the U.S.

### Uses of Thin Films

The thin films of the present invention are well suited for many uses. The high degree of uniformity of the components of the film makes them particularly well suited for incorporating pharmaceuticals. Furthermore, the polymers used in construction of the films may be chosen to allow for a range of disintegration times for the films. A variation or extension in the time over which a film will disintegrate may achieve control over the rate that the active is released, which may allow for a sustained release delivery system. In addition, the films may be used for the administration of an active to any of several body surfaces, especially those including mucous membranes, such as oral, anal, vaginal, ophthalmological, the surface of a wound, either on a skin surface or within a body such as during surgery, and similar surfaces.

The films may be used to orally administer an active. This is accomplished by preparing the films as described above and introducing them to the oral cavity of a mammal. This film may be prepared and adhered to a second or support layer from which it is removed prior to use, i.e. introduction to the oral cavity. An adhesive may be used to attach the film to the support or backing material which may be any of those known in the art, and is preferably not water soluble. If an adhesive is used, it will desirably be a food grade adhesive that is ingestible and does not alter the properties of the active. Mucoadhesive compositions are particularly useful. The film compositions in many cases serve as mucoadhesives themselves.

The films may be applied under or to the tongue of the mammal. When this is desired, a specific film shape, corresponding to the shape of the tongue may be preferred. Therefore the film may be cut to a shape where the side of the film corresponding to the back of the tongue will be longer than the side corresponding to the front of the tongue. Specifically, the desired shape may be that of a triangle or trapezoid. Desirably, the film will adhere to the oral cavity preventing it from being ejected from the oral cavity and permitting more of the active to be introduced to the oral cavity as the film dissolves.

Another use for the films of the present invention takes advantage of the films' tendency to dissolve quickly when introduce to a liquid. An active may be introduced to a liquid by preparing a film in accordance with the present invention, introducing it to a liquid, and allowing it to dissolve. This may be used either to prepare a liquid dosage form of an active, or to flavor a beverage.

The films of the present invention are desirably packaged in sealed, air and moisture resistant packages to protect the active from exposure oxidation, hydrolysis, volatilization and interaction with the environment. Referring to FIG. 1, a packaged pharmaceutical dosage unit 10, includes each film 12 individually wrapped in a pouch or between foil and/or plastic laminate sheets 14. As depicted in FIG. 2, the pouches 10, 10′ can be linked together with tearable or perforated joints 16. The pouches 10, 10′ may be packaged in a roll as depicted in FIG. 5 or stacked as shown in FIG. 3 and sold in a dispenser 18 as shown in FIG. 4. The dispenser may contain a full supply of the medication typically prescribed for the intended therapy, but due to the thinness of the film and package, is smaller and more convenient than traditional bottles used for tablets, capsules and liquids. Moreover, the films of the present invention dissolve instantly upon contact with saliva or mucosal membrane areas, eliminating the need to wash the dose down with water.

Desirably, a series of such unit doses are packaged together in accordance with the prescribed regimen or treatment, e.g., a 10-90 day supply, depending on the particular therapy. The individual films can be packaged on a backing and peeled off for use.

The features and advantages of the present invention are more fully shown by the following examples which are provided for purposes of illustration, and are not to be construed as limiting the invention in any way.

### EXAMPLES

### Example 1

### Pharmacokinetic Study in Göttingen Minipigs

### Dosing Regimen

Göttingen minipigs (4 males and 2 females) were fasted overnight and then dosed with (1) a 10 mg rizatriptan film, (2) 2×5 mg rizatriptan films via sublingual or buccal application or (3) a 10 mg MAXALT® (Rizatriptan) oral tablet. Plasma samples were to be collected from each animal at pre-dose and at 5, 15, 30, 45, 60, 75, 90, 105, 120 and 150 minutes and 3, 3.5, 4, 5, 6 and 8 hours post dosing via a previously implanted venous access port (VAP). However, actual sample collection may have varied slightly from the protocol depending on the behavior of each animal or the performance of the VAPs. Samples were then extracted and analyzed for rizatriptan content using a qualified bioanalytical LC-MS/MS method.

### Test Samples

Samples of the 10 mg MAXALT tablet (Doses 1 and 4) were purchased commercially. The composition of each rizatriptan film test article is summarized in Table 1:

TABLE 1
Formulation Composition of Rizatriptan Film Test Articles

| | Rizatriptan | | | | |
|---|---|---|---|---|---|
| | 5 mg | 5 mg | 10 mg | 10 mg | 10 mg |
| Ingredient/Property | Dose 2 | Dose 3 | Dose 5 | Dose 6 | Dose 7 and 8 |
| HPMC E15 | 24.29% | 12.75% | 24.29% | 22.44% | 24.29% |
| | (4.858 mg) | (1.9125 mg) | (9.716 mg) | (17.945 mg) | (9.716 mg) |
| PEO WSR N80 LEO | 12.14% | 25.567 | 12.14% | | 12.14% |
| | (2.428 mg) | (3.835 mg) | (4.856 mg) | | (4.856 mg) |
| Maltitol (Added as | 12.14% | 6.38% | 12.14% | 8.973% | 12.14% |
| Lycasin 80/55) | (2.428 mg) | (0.957 mg) | (4.856 mg) | (7.178 mg) | (4.856 mg) |
| Plasdone K29/32 | | | | 40.38% | |
| (PVP) ISP | | | | (32.307 mg) | |
| Glycerin | | 6.37% | | 2.993% | |
| | | (0.9555 mg) | | (2.3944 mg) | |
| Sucralose | 7.00% | | 7.00% | 3.50% | 7.00% |
| | (1.400 mg) | | (2.800 mg) | (2.800 mg) | (2.800 mg) |
| Peceol | | 0.500% | | | |
| | | (0.075 mg) | | | |
| Rizatriptan Benzoate | 36.33% | 48.433% | 36.33% | 18.17% | 36.33% |
| | (7.266 mg) | (7.265 mg) | (14.532 mg) | (14.532 mg) | (14.532 mg) |
| Peppermint 2303 | 6.00% | | 6.00% | 3.00% | 6.00% |
| Flavor | (1.200 mg) | | (2.400 mg) | (2.4o0 mg) | (2.400 mg) |
| Butylated | 0.10% | | 0.10% | 0.050% | 0.10% |
| Hydroxytoluene | (0.020 mg) | | (0.040 mg) | (0.040 mg) | (0.040 mg) |
| Titanium Dioxide | 1.00% | | 1.00% | | 1.00% |
| | (0.200 mg) | | (0.400 mg) | | (0.400 mg) |
| Menthol FP 4594 | 1.00% | | 1.00% | 0.050% | 1.00% |
| Flavor | (0.200 mg) | | (0.400 mg) | (0.400 mg) | (0.400 mg) |
| % Solids | 30 | 40 | 30 | 50 | 30.0 |
| % Moisture | 0.96 | 1.36 | 2.33 | 3.00 | 2.33 |
| Dry Target Strip wt. | 20 mg | 15 mg | 40 mg | 80 mg | 40 mg |
| Target Strip Wt. | 20.194 mg | 15.207 mg | 40.954 mg | 82.477 mg | 40.954 mg |
| Accounting for % | | | | | |
| Moisture | | | | | |
| Strip Wt. Range | 19-21 mg | 14-16 mg | 39-42 mg | 84-87 mg | 38-39 mg |
| Strip Size (mm × | 13 × 22 | 7 × 18 Active* | 26 × 22 | 26 × 22 | 25.4 × 22.2 |
| mm) | | 11 × 22 Occlusive | | | |

*Dose 3 was of a two layer drug product in which a smaller, fast dissolving (7 mm × 18 mm) containing rizatriptan active film was prepared first and then laminated into a larger, slower dissolving (11 mm × 22 mm) occlusive film. The occlusive film was prepared from the following ingredients; (1) 7.48% PEO WSR 1105 LEO, (2) 51.40% PEO WSR N80 LEO, (3) 12.15% Maltitol, (4) 12.15% Glycerin, (5) 10.28% HPMC, (6) 2.00% Sucralose, (7) 4.00% peppermint 2303, (8) 0.50% glyceryl monooleate and (9) 0.04% Blue #1. All other film formulations were a single-layer drug product containing the Active. Occlusive film was produced using same general process as described below.

### Procedure for Preparing Rizatriptan Formulations (Doses 2, 3, 5, 6, 7, 8)

The maltitol syrup (Lycasin 80/55), menthol flavor FP 4594, titanium dioxide, and water were added to a fabricated glass bowl. A solution of the butylated hydroxytoluene (BHT) and peppermint 2303 flavor was then were added to the glass bowl. A blend of rizatriptan benzoate, sucralose, and polymers (HPMC and PEO or HPMC and Plasdone K 29/32 (PVP)) was added to the bowl. The bowl was equipped with a Variac controlled heating mantel and the heat was turned on. The solution was prepared as described below using the Degussa Dental Multivac Compact, and the following steps:

| | | | | |
|---|---|---|---|---|
| 1) | 8 Minutes | Stirring = 125 rpm | Vacuum = 0% | Temperature = |
| | | | | 60° C. |
| 2) | 20 Minutes | Stirring = 125 rpm | Vacuum = 0% | Temperature = |
| | | | | 40° C. |

| 3) | Cut off heat and the heating mantel removed | | |
|---|---|---|---|
| 4) | Distilled water was added to compensate for water lost | | |
| 5) | 40 Minutes | Stirring = 125 rpm | Vacuum = 60% (18.5 in Hg) |
| 6) | 40 Minutes | Stirring = 125 rpm | Vacuum = 90% (26 in Hg) |
| 7) | 20 Minutes | Stirring = 125 rpm | Vacuum = 95% (27 in Hg) |
| 8) | 8 Minutes | Stirring = 125 rpm | Vacuum = 98% (28 in Hg) |
| 9) | 4 Minutes | Stirring = 125 rpm | Vacuum = 100% (29 in Hg) |
| 10) | Distilled water was added to compensate for water lost | | |
| 11) | 8 Minutes | Stirring = 125 rpm | Vacuum = 100% (29 in Hg) |

Alternatively, the rizatriptan film can be prepared using commercial-scale equipment. For example, a 35 kilogram batch of the drug product is prepared by mixing the ingredients in a heated 12 gallon mixer. The mixture is transferred to a 12 gallon hold tank from where it is pumped to the coating head of an Olbrich film coater. The film is coated, dried and sealed in the primary packaging using a Medipharm Doyen commercial packager.

The solution was cast into wet film using a K-Control Coater with the micrometer adjustable wedge bar set at 320 microns onto Mylar substrate. The film was dried 17 minutes in an 80° C. convection air oven. The sheets of film were cut into either 0.875×0.5 inch strips to yield a 5 mg dose of rizatriptan base or 0.875×1 inch strips to yield a 10 mg dose of rizatriptan base.

### Clinical Signs

All animals were observed for mortality and general condition twice daily. On each treatment day, all animals were observed once prior to and once after administration of the test article. Overall, there were no clinical signs after dosing or changes in body weight that were considered test-article related.

### Plasma Rizatritan Levels

Plasma rizatriptan levels for all test articles were determined on the schedule set forth above. The pharmacokinetic profiles of the 10 mg MAXALT® oral tablet (Doses 1 and 4) and the 10 mg rizatriptan film (10 mg Rizatriptan Film; Doses 5 and 8) are illustrated in FIGS. 38 and 39, respectively.

A comparison of the MAXALT® PK curves to the PK curves for the preferred film demonstrates that the film products of the present invention release rizatriptan into the systemic circulation earlier than MAXALT® tablets. This finding is illustrated by comparing the average PK profile of each dosage form, FIG. 40.

PK data for one of the MAXALT treated pigs was excluded because the results indicated that the PK profile for that animal was not representative of the performance of the MAXALT tablet in this animal model.

Moreover, evaluation of the average pharmacokinetic curves shows significantly different T_{MAX} and C_{MAX} values; however, similar exposure (AUC) for each dosage form Table 2.

TABLE 2
Pharmacokinetic Parameters for 10 mg MAXALT Oral Tablet and
10 mg Rizatriptan Sublingual Film

| | T_{MAX} | C_{MAX} | AUC₀₋₃₀₀ |
|---|---|---|---|
| Dosage Form | (minutes) | (ng/mL) | ng · min/mL |
| Tablet | 150 | 22.9 | 2590.8 |
| Film | 60 | 16.0 | 2087.9 |

Thus, the Tmax for the film product was 60% faster and had comparable Cmax and AUC

The shorter T_{MAX} for the film is likely a result of sublingual absorption of rizatriptan. It is known that drugs can be absorbed through the sublingual mucosa thereby allowing for direct access to the systemic circulation without requiring transit/absorption through the gastrointestinal tract (avoiding “first pass” effects). More importantly, both dosage forms exhibit similar drug exposure as evidenced by comparable AUC values. Thus, the delivery of 10 mg rizatriptan via a sublingual film poses no greater risk than is incurred through exposure to the mg tablet.

### Example 2

### Pharmacokinetic Study in Humans

The relative bioavailability of 10 mg rizatriptan sublingual film of the present invention was compared to that of 10 mg Maxalt-MLT Orally Disintegrating Tablets (ODT) by Merck & Co., Inc. following a single oral dose in healthy adult subjects when administered under fasted conditions.

Methodology

This was an open-label, single-dose, randomized, 2-period, 2-treatment crossover study under fasted conditions of 12 human subjects. The total duration of the study, screening through study exit, was approximately 5 weeks with at least a 7-day washout period between doses. At study check-in, the subjects reported to the clinical site at least 10 hours prior to Day 1 dosing and were required to stay for 12 hours after Day 1 dosing. Blood sample collections were obtained within 90 minutes prior to dosing (0 hour) and after dose administration at 5, 10, 15, 20, 25, 30, and 45 minutes and at 1.0, 1.25, 1.5, 1.75, 2, 2.5, 3, 3.5, 4, 5, 6, 8, and 12 hours. A total of 21 blood samples were collected per study period for a total of 42 samples or 294 mL total volume per subject.

Samples of the 10 mg MAXALT-MLT tablet were purchased commercially. The composition of each rizatriptan film test article is as described above in Example 1 dose 8.

At dosing, for those subjects receiving the film product of the invention the sublingual film was placed immediately under the tongue, close to the base either on the left or the right side of the center. The subjects were instructed to keep the film in place until completely dissolved whereupon it was swallowed with saliva. The subjects indicated when they felt that the film had dissolved and the clinical staff performed a mouth check to confirm complete dissolution. If the dissolution was not complete, this process was repeated approximately every 30 seconds until complete dissolution. While the test film was dissolving, the subjects were advised not to chew, swallow, or talk.

At dosing, for those subjects receiving the MAXALT-MLT tablets the ODT was placed on the tongue, where it dissolved completely and was swallowed with the saliva. The subjects were instructed to keep the ODT in place until completely dissolved whereupon it was swallowed with saliva. The subjects indicated when they felt that the tablet had dissolved and the clinical staff performed a mouth check to confirm complete dissolution. If the dissolution was not complete, this process was repeated approximately every 30 seconds until complete dissolution. While the ODT was dissolving, the subjects were advised not to chew, swallow, or talk.

### Pharmacokinetic Results

FIG. 41 shows the mean plasma rizatriptan concentration on a linear scale, from time 0-12 hours after dosing.

Table 3 summarizes the geometric means, ratios of means, and 90% CIs of ln-transformed rizatriptan data for Test Product A versus Reference Product B.

TABLE 34

| | Geometric Means* | | | |
|---|---|---|---|---|
| | Rizatriptan | Maxalt- | | |
| | Sublingual | MLT, | | 90% CI |
| | Film, | ODT | | (Lower Limit, |
| Parameter | 10 mg | 10 mg | % Ratio | Upper Limit) |
| AUC₀₋ₜ (ng · h/mL) | 69.32 | 70.89 | 97.78 | (84.60, 113.01) |
| AUCReftₘₐₓ | 20.30 | 15.41 | 131.79 | (99.82, 173.98) |
| (ng · h/mL) | | | | |
| AUC_{0-inf} (ng · h/mL) | 70.93 | 72.71 | 97.54 | (84.40, 112.74) |
| Cₘₐₓ (ng/mL) | 21.50 | 20.52 | 104.75 | (86.27, 127.20) |

For rizatriptan, the Tmax was no more than 30 minutes for the film test article and maintained until about 2 hours. The Tmax was about 2.0 hours for the MAXALT®-MLT and quickly diminished. Accordingly, one would expect the film product to produce a faster onset of relief as compared to MAXALT®-MLT.

Moreover, the PK and statistical results of this study indicate that the test/reference ratio of geometric means for ln-transformed AUC₀₋ₜ was 97.78% (90% CI 84.60%-113.01%), for ln-transformed AUCReftₘₐₓ was 131.79% (90% CI 99.82%-173.98%), for ln-transformed AUC_{0-inf} was 97.54% (90% CI 84.40%-112.74%), and for ln-transformed Cₘₐₓ was 104.75% (90% CI 86.27%-127.20%).

### Example 3

The following example is based on applicants' manufacture of 73 lots of film (Lots 1-73) containing approximately 2,000,000 individual dosage units per lot. They were manufactured: (i) for commercial use and regulatory approval; and (ii) in compliance with FDA standards and regulations, including those relating to analytical chemical testing for variation in active in individual dosage units.

The films were manufactured in accordance with the present invention by a process comprising: (a) forming a flowable polymer matrix comprising a water-soluble polymer, a solvent and a pharmaceutical active, said matrix having a substantially uniform distribution of said active; (b) casting said flowable polymer matrix; (c) controlling drying through a process comprising conveying said polymer matrix through a drying apparatus and evaporating at least a portion of said solvent to form a visco-elastic film, having said active substantially uniformly distributed throughout, within about the first 4 minutes by rapidly increasing the viscosity of said polymer matrix upon initiation of drying to maintain said substantially uniform distribution of said active by locking-in or substantially preventing migration of said active within said visco-elastic film wherein the polymer matrix temperature is 100° C. or less; (d) forming the pharmaceutical film from said visco-elastic film, wherein said resulting pharmaceutical film has a water content of 10% or less and said substantially uniform distribution of active by said locking-in or substantially preventing migration of said active is maintained, such that uniformity of content in the amount of the active in substantially equal sized individual dosage units, sampled from different locations of said pharmaceutical film, varies by no more than 10%; and (e) performing analytical chemical tests for uniformity of content of said active in substantially equal sized individual dosage units of said sampled resulting pharmaceutical films; (f) repeating steps (a) through (e) in order to manufacture additional films.

Ten (10) individual dosage units all having the same dimensions were cut out from different locations of each of the 73 lots of resulting films using a commercial packaging machine, thus providing 730 randomly sampled individual dosage units, ten each from the 73 separate lots. All samples were analyzed by a validated method, in compliance with FDA guidelines and regulations regarding same, using analytical chemical testing, in which the pharmaceutical active was extracted and analyzed by High Performance Liquid Chromatography (HPLC) against an external standard to quantify the amount of active present in each individual dosage unit.

Tables 3-A, 4-B and 4-C reflect the uniformity of content of active of individual dosage units within particular lots and across the 73 different lots.

TABLE 4-C

| | |
|---|---|
| Lots less than 5% | lots 5% to 10% |

| Lot # | % Difference | Lot # | % Difference |
|---|---|---|---|
| 24 | 2.0% | 10 | 5.0% |
| 45 | 2.6% | 25 | 5.0% |
| 17 | 2.8% | 39 | 5.0% |
| 21 | 2.8% | 41 | 5.2% |
| 22 | 3.1% | 13 | 5.2% |
| 16 | 3.1% | 35 | 5.3% |
| 60 | 3.2% | 5 | 5.4% |
| 50 | 3.4% | 63 | 5.5% |
| 72 | 3.4% | 34 | 5.5% |
| 33 | 3.6% | 38 | 5.6% |
| 43 | 3.6% | 40 | 5.6% |
| 19 | 3.7% | 73 | 5.7% |
| 46 | 3.8% | 7 | 5.8% |
| 29 | 3.9% | 8 | 5.9% |
| 2 | 3.9% | 6 | 6.2% |
| 4 | 4.0% | 11 | 6.3% |
| 61 | 4.0% | 55 | 6.3% |
| 30 | 4.0% | 69 | 6.7% |
| 48 | 4.1% | 3 | 6.7% |
| 15 | 4.1% | 12 | 6.7% |
| 52 | 4.2% | 70 | 7.1% |
| 54 | 4.2% | 32 | 7.4% |
| 51 | 4.2% | 49 | 7.8% |
| 44 | 4.3% | 27 | 8.2% |
| 62 | 4.3% | 64 | 8.3% |
| 56 | 4.3% | 57 | 8.9% |
| 31 | 4.4% | 37 | 9.5% |
| 28 | 4.4% | | |
| 14 | 4.4% | | |
| 68 | 4.4% | | |
| 42 | 4.4% | | |
| 18 | 4.4% | | |
| 66 | 4.5% | | |
| 47 | 4.5% | | |
| 23 | 4.6% | | |
| 20 | 4.6% | | |
| 9 | 4.6% | | |
| 58 | 4.6% | | |
| 65 | 4.7% | | |
| 26 | 4.8% | | |
| 53 | 4.8% | | |
| 36 | 4.8% | | |
| 1 | 4.9% | | |
| 59 | 4.9% | | |
| 67 | 4.9% | | |
| 71 | 4.9% | | |
| total | 46 | total | 27 |

In accordance with this example, first, the uniformity of content of active in a lot is determined through establishing the amount of active (A_{N(i)}) actually present in each sampled individual dosage unit from the same lot (N) as determined by taking the difference between the amount of active in the sample with the most active (Max_{LOT(N)}) minus the amount of active in the sample with the least amount of active (Min_{LOT(N)}) and dividing the difference by the average amount of active in the lot samples (Lot_{(N)} Sample Average). That is: (Max_{LOT(N)}−Min_{LOT(N)})/((A_{N(1)+})A_{N(2)+++}A_{N(10)})/10). The results are shown in Table 4-A.

Second, the uniformity of content across different lots is determined through establishing the amount of active actually present in each sampled individual dosage unit from all 73 lots and comparing that amount of active with a “target” or “desired” amount of active contained therein. The target amount of active, when it is a pharmaceutical, is referred to as the “Label Claim”, thus identifying the amount of pharmaceutical active in the film to a user. The desired amount is 100% of the target amount. Each individual dosage unit film cut from any individual lot must have the desired content of pharmaceutical active, varying no more that 10% from the target or desired amount. See Table 4-B.

The results shown in Tables 4-A, 4-B and 4-C indicate that the films produced by the present invention have the required uniformity of content based on analytical chemical testing. First, the amount of active varies by no more than 10% between individual dosage units sampled from a particular lot of resulting film. See Table 4-A. Second, the amount of active across different lots of resulting film varies no more than 10% from the desired amount of the active. See Table 4-B. Finally, the uniformity of content of the 73 lots of film meets even more stringent standards, for example, the data shows: (i) 46 lots of film wherein the uniformity of content of active is shown with the amount of active varying by less than 5%; (ii) 15 lots of film wherein the uniformity of content of active is shown with the amount of active varying by less than 4%; 4 lots of film wherein the uniformity of content of active is shown with the amount of active varying by less than 3%; and 1 lot of film wherein the uniformity of content of active is shown with the amount of active varying by only 2%. See Table 4-C.

While there have been described what are presently believed to be the preferred embodiments of the invention, those skilled in the art will realize that changes and modifications may be made thereto without departing from the spirit of the invention, and it is intended to include all such changes and modifications as fall within the true scope of the invention.

What is claimed is:

1. A film product comprising:
a. a polymer component; and
b. a therapeutically effective amount of rizatriptan or a pharmaceutically acceptable salt thereof.

2. The film product according to claim 1, wherein the rizatriptan or a pharmaceutically acceptable salt thereof is rizatriptan benzoate.

3. The film product according to claim 1, wherein the polymer component comprises polyethylene oxide and hydroxypropyl methyl cellulose.

4. The film product according to claim 1, wherein the film product is a dosage unit and the amount of rizatriptan or a pharmaceutically acceptable salt thereof is equivalent to about 10 milligrams of rizatriptan base.

5. The film product according to claim 1, wherein the film product is a dosage unit and the amount of rizatriptan or a pharmaceutically acceptable salt thereof is equivalent to about 5 milligrams of rizatriptan base.

6. The film product according to claim 1, wherein the film product is a dosage unit and the amount of rizatriptan or a pharmaceutically acceptable salt thereof is equivalent to about 15 milligrams of rizatriptan base or less.

7. The film product according to claim 1, wherein the film product is a dosage unit and the amount of rizatriptan or a pharmaceutically acceptable salt thereof is equivalent to about 10 milligrams of rizatriptan base or less.

8. The film product according to claim 1, wherein the film product is a dosage unit and the amount of rizatriptan or a pharmaceutically acceptable salt thereof is equivalent to about 5 milligrams of rizatriptan base or less.

9. The film product according to claim 1, wherein the film product is a dosage unit and the amount of rizatriptan or a pharmaceutically acceptable salt thereof is equivalent to about 5 to about 15 milligrams of rizatriptan base.

10. The film product according to claim 1, wherein the film product is a dosage unit and the amount of rizatriptan or a pharmaceutically acceptable salt thereof is equivalent to about 5 to about 10 milligrams of rizatriptan base.

11. The film product according to claim 2, wherein the film product is a dosage unit and contains about 14.5 milligrams of rizatriptan benzoate.

12. The film product according to claim 2, wherein the film product is a dosage unit and contains about 7.25 milligrams of rizatriptan benzoate.

13. The film product of claim 1, wherein the film product is a unit dosage form.

14. The film product of claim 13, wherein the unit dosage form is configured such that when the unit dosage form is administered to human subjects in a fasted state, the time to reach maximum plasma concentration (Tₘₐₓ) of rizatriptan is about 10 minutes to about 60 minutes after administration of the dosage form.

15. The film product of claim 13, wherein the unit dosage form is configured such that when the unit dosage form is administered to human subjects in a fasted state, the time to reach maximum plasma concentration (Tₘₐₓ) of rizatriptan is about 15 minutes to about 45 minutes after administration of the dosage form.

16. The film product of claim 13, wherein the unit dosage form is configured such that when the unit dosage form is administered to human subjects in a fasted state, the time to reach maximum plasma concentration (Tₘₐₓ) of rizatriptan is about 30 minutes or less.

17. The film product of claim 13, wherein the unit dosage form is configured such that the unit dosage form contains about 14.5 milligrams of rizatriptan benzoate and provides a mean plasma concentration over 0-12 hours (AUC₀₋₁₂) of about 35 to about 150 ng·hr/mL of rizatriptan after oral administration of a single dosage form to human subjects in a fasted state.

18. The film product of claim 13, wherein the unit dosage form is configured such that the unit dosage form contains about 14.5 milligrams of rizatriptan benzoate and provides a mean plasma concentration over 0-12 hours (AUC₀₋₁₂) of about 56.7 to about 88.6 ng·hr/mL of rizatriptan after oral administration of a single dosage form to human subjects in a fasted state.

19. The film product of claim 13, wherein the unit dosage form is configured such that the unit dosage form contains about 14.5 milligrams of rizatriptan benzoate and provides a mean plasma concentration over 0-12 hours (AUC₀₋₁₂) of about 60 to about 80 ng·hr/mL of rizatriptan after oral administration of a single dosage form to human subjects in a fasted state.

20. The film product of claim 13, wherein the unit dosage form is configured such that the unit dosage form contains about 14.5 milligrams of rizatriptan benzoate and provides a mean plasma concentration over 0-12 hours (AUC₀₋₁₂) of about 71 ng·hr/mL of rizatriptan after oral administration of a single dosage form to human subjects in a fasted state.

21. The film product of claim 13, wherein the unit dosage form is configured such that the unit dosage form contains about 7.25 milligrams of rizatriptan benzoate and provides a mean plasma concentration over 0-12 hours (AUC₀₋₁₂) of about 17.5 to about 75 ng·hr/mL of rizatriptan after oral administration of a single dosage form to human subjects in a fasted state.

22. The film product of claim 13, wherein the unit dosage form is configured such that the unit dosage form contains about 7.25 milligrams of rizatriptan benzoate and provides a mean plasma concentration over 0-12 hours (AUC₀₋₁₂) of about 28 to about 45 ng·hr/mL of rizatriptan after oral administration of a single dosage form to human subjects in a fasted state.

23. The film product of claim 13, wherein the unit dosage form is configured such that the unit dosage form contains about 7.25 milligrams of rizatriptan benzoate and provides a mean plasma concentration over 0-12 hours (AUC₀₋₁₂) of about 30 to about 40 ng·hr/mL of rizatriptan after oral administration of a single dosage form to human subjects in a fasted state.

24. The film product of claim 13, wherein the unit dosage form is configured such that the unit dosage form contains about 7.25 milligrams of rizatriptan benzoate and provides a mean plasma concentration over 0-12 hours (AUC₀₋₁₂) of about 35.5 ng·hr/mL of rizatriptan after oral administration of a single dosage form to human subjects in a fasted state.

25. The film product of claim 13, further comprising ondansetron or a salt thereof.

26. A film product comprising:
a. a polymer component comprising polyethylene oxide and hydroxypropyl methyl cellulose; and
b. a therapeutically effective amount of rizatriptan benzoate.

27. A film dosage unit product comprising:
a. a polymer component comprising polyethylene oxide and hydroxypropyl methyl cellulose;
b. about 14.5 or about 7.25 milligrams of rizatriptan benzoate.

28. The film dosage unit of claim 27, wherein the unit dosage form is configured such that when the unit dosage form is administered to human subjects in a fasted state, the time to reach maximum plasma concentration (Tₘₐₓ) of rizatriptan is about 10 minutes to about 60 minutes after administration of the dosage form.

29. The film dosage unit of claim 27, wherein the unit dosage form is configured such that when the unit dosage form is administered to human subjects in a fasted state, the time to reach maximum plasma concentration (Tₘₐₓ) of rizatriptan is about 15 minutes to about 45 minutes after administration of the dosage form.

30. The film dosage unit of claim 27, wherein the unit dosage form is configured such that when the unit dosage form is administered to human subjects in a fasted state, the time to reach maximum plasma concentration (Tₘₐₓ) of rizatriptan is about 30 minutes or less.

31. The film product of claim 1, further comprising a penetration enhancer.

32. The film product of claim 31, wherein the penetration enhancer is selected from the group consisting of sodium lauryl Sulfate, Sodium decyl sulfate, Sodium octyl sulfate, Sodium laureth sulfate, N-Lauryl sarcosinate, Cetyltrimethyl ammonium bromide, Decyltrimethyl ammonium bromide, Benzyldimethyl dodecyl ammonium chloride, Myristyltrimethyl ammonium chloride, Dodecyl pyridinium chloride, Decyldimethyl ammonio propane sulfonate, Myristyldimethyl ammonlo propane sulfonate, Palmityldimethyl ammonio propane sulfonate, ChemBetaine CAS, ChemBctainc Oleyl, Palmltoyl camitine chloride, Nonylphenoxypolyoxyethylene, Polyoxyethylene sorbitan monolaurate, Polyoxyethylene sorbitan monopalmitate, Sorbitan monooleate, Triton-X 100, Sodium deoxycholate, Sodium glycocholate, Cholic acid, Hexanoic acid, Heptanoic acid, Methyllaurate, Isopropyl myrlstate, Isopropyl palmitate, Methyl palmitate, Diethyl sebaccate, Sodium oleate, Urea, Lauryl amine, Caprolactam, Methyl pyrrolidone, Octyl pyrrolidone, Methyl piperazine, Phenyl piperazine, Sodium salicylate, Carbopol934P, Glyccyrhetinic acid, Bromelain, Pinene oxide, Limonene, Cineole, Octyl dodecanol, Fenchone, Menthane, Trimethoxy propylene methyl benzene, and combinations thereof.

33. The film product of claim 26, further comprising a penetration enhancer.

34. The film product of claim 33, wherein the penetration enhancer is selected from the group consisting of sodium lauryl Sulfate, Sodium decyl sulfate, Sodium octyl sulfate, Sodium laureth sulfate, N-Lauryl sarcosinate, Cetyltrimethyl ammonium bromide, Decyltrimethyl ammonium bromide, Benzyldimethyl dodecyl ammonium chloride, Myristyltrimethyl ammonium chloride, Dodecyl pyridinium chloride, Decyldimethyl ammonio propane sulfonate, Myristyldimethyl ammonlo propane sulfonate, Palmityldimethyl ammonio propane sulfonate, ChemBetaine CAS, ChemBctainc Oleyl, Palmltoyl camitine chloride, Nonylphenoxypolyoxyethylene, Polyoxyethylene sorbitan monolaurate, Polyoxyethylene sorbitan monopalmitate, Sorbitan monooleate, Triton-X 100, Sodium deoxycholate, Sodium glycocholate, Cholic acid, Hexanoic acid, Heptanoic acid, Methyllaurate, Isopropyl myrlstate, Isopropyl palmitate, Methyl palmitate, Diethyl sebaccate, Sodium oleate, Urea, Lauryl amine, Caprolactam, Methyl pyrrolidone, Octyl pyrrolidone, Methyl piperazine, Phenyl piperazine, Sodium salicylate, Carbopol934P, Glyccyrhetinic acid, Bromelain, Pinene oxide, Limonene, Cineole, Octyl dodecanol, Fenchone, Menthane, Trimethoxy propylene methyl benzene, and combinations thereof.

35. The film dosage unit of claim 27, further comprising a penetration enhancer.

36. The film dosage unit of claim 35, wherein the penetration enhancer is selected from the group consisting of sodium lauryl Sulfate, Sodium decyl sulfate, Sodium octyl sulfate, Sodium laureth sulfate, N-Lauryl sarcosinate, Cetyltrimethyl ammonium bromide, Decyltrimethyl ammonium bromide, Benzyldimethyl dodecyl ammonium chloride, Myristyltrimethyl ammonium chloride, Dodecyl pyridinium chloride, Decyldimethyl ammonio propane sulfonate, Myristyldimethyl ammonlo propane sulfonate, Palmityldimethyl ammonio propane sulfonate, ChemBetaine CAS, ChemBctainc Oleyl, Palmltoyl camitine chloride, Nonylphenoxypolyoxyethylene, Polyoxyethylene sorbitan monolaurate, Polyoxyethylene sorbitan monopalmitate, Sorbitan monooleate, Triton-X 100, Sodium deoxycholate, Sodium glycocholate, Cholic acid, Hexanoic acid, Heptanoic acid, Methyllaurate, Isopropyl myrlstate, Isopropyl palmitate, Methyl palmitate, Diethyl sebaccate, Sodium oleate, Urea, Lauryl amine, Caprolactam, Methyl pyrrolidone, Octyl pyrrolidone, Methyl piperazine, Phenyl piperazine, Sodium salicylate, Carbopol934P, Glyccyrhetinic acid, Bromelain, Pinene oxide, Limonene, Cineole, Octyl dodecanol, Fenchone, Menthane, Trimethoxy propylene methyl benzene, and combinations thereof.
